(19) United States
(12) Patent Application Publication
(10) Pub. No.: US 2025/0048919 A1
(43) Pub. Date: Feb. 6, 2025

(54) FUNCTIONAL LAYER MATERIAL, LIGHT-EMITTING DEVICE, LIGHT-EMITTING SUBSTRATE AND LIGHT-EMITTING APPARATUS

(71) Applicant: BOE TECHNOLOGY GROUP CO., LTD., Beijing (CN)

(72) Inventors: Rongrong GAO, Beijing (CN); Dan WANG, Beijing (CN); Lei CHEN, Beijing (CN)

(73) Assignee: BOE TECHNOLOGY GROUP CO., LTD., Beijing (CN)

(21) Appl. No.: 18/710,615

(22) PCT Filed: Jul. 18, 2023
(86) PCT No.: PCT/CN2023/107939

§ 371 Date: May 16, 2024

(30) Foreign Application Priority Data
Jul. 27, 2022 (CN) 202210890528.5

(51) Int. Cl. H10K 85/60; C07C 211/60; C07C 211/61; C07D 251/24; C07D 311/96; C07D 401/14; C07D 405/04; C07D 405/10; C07F 9/46; C07F 9/6521; H10K 50/13; H10K 50/19

(52) U.S. Cl. CPC H10K 85/633; C07C 211/60; C07C 211/61; C07D 251/24; C07D 311/96; C07D 401/14; C07D 405/04; C07D 405/10; C07F 9/46; C07F 9/6521; H10K 85/636; H10K 85/654; C07C 2603/18; C07C 2603/54; C07C 2603/74; H10K 50/13; H10K 50/19; H10K 85/615; H10K 85/624; H10K 85/626; H10K 85/6574

(57) ABSTRACT

A functional layer material includes a compound centered on an sp3 hybridized carbon atom which includes a first type of compound. The first type of compound is selected from any of structures represented by a general formula (I), wherein at least one of a, b, m and n is not 0; A1 and A2 are independently selected from any of a substituted or unsubstituted trivalent aryl group, fused ring trivalent aryl group and fused ring trivalent heteroaryl group; B1 and B2 are independently selected from any of a substituted or unsubstituted alkylene group, arylene group and heteroarylene group; L1 to L4 are independently selected from any of a single bond, a substituted or unsubstituted phenylene group and biphenylene group; and Ar1 to Ar8 are independently selected from any one of a substituted or unsubstituted alkyl group, aryl group, heteroaryl group, fused ring aryl group and fused ring heteroaryl group.

### CROSS-REFERENCE TO RELATED APPLICATIONS

This application is a national phase entry under 35 USC 371 of International Patent Application No. PCT/CN2023/107939, filed on Jul. 18, 2023, which claims priority to Chinese Patent Application No. 202210890528.5, filed on Jul. 27, 2022, which are incorporated herein by reference in their entirety.

### TECHNICAL FIELD

The present disclosure relates to the field of display and lighting technologies, and in particular, to a functional layer material, a light-emitting device, a light-emitting substrate and a light-emitting apparatus.

### BACKGROUND

Organic light-emitting diode (OLED) light-emitting apparatuses have become the most promising new light-emitting apparatuses in recent years due to self-luminescence, high luminous efficiency, and other advantages. During light emission of the OLED light-emitting apparatus, holes from an anode and electrons from a cathode are emitted to a light-emitting layer included in the OLED light-emitting apparatus. These electrons and holes are combined to produce electron-hole pairs, and the produced electron-hole pairs are converted from a singlet state to a ground state to emit light.

### SUMMARY

In an aspect, a functional layer material is provided. The functional layer material includes a compound centered on an sp3 hybridized carbon atom. The compound centered on the sp3 hybridized carbon atom includes a first type of compound, and the first type of compound is selected from any one of structures shown in a following general formula (I).

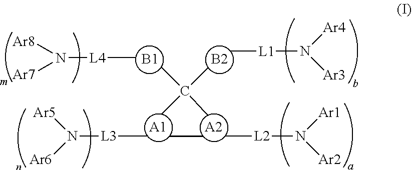

Where values of a, b, m and n are each independently selected from any one of 0, 1, 2, 3 and 4, and at least one of a, b, m and n is not 0.

A1 and A2 are same or different, and are each independently selected from any one of a substituted or unsubstituted trivalent aryl group, a substituted or unsubstituted fused ring trivalent aryl group, and a substituted or unsubstituted fused ring trivalent heteroaryl group.

B1 and B2 are same or different, and are each independently selected from any one of a substituted or unsubstituted alkylene group, a substituted or unsubstituted arylene group, and a substituted or unsubstituted heteroarylene group.

L1, L2, L3 and L4 are same or different, and are each independently selected from any one of a single bond, a substituted or unsubstituted phenylene group, a substituted or unsubstituted biphenylene group, a substituted or unsubstituted terphenylene group, a substituted or unsubstituted fluorenyl group, a substituted or unsubstituted adamantyl group, and a substituted or unsubstituted heteroarylene group.

Ar1, Ar2, Ar3, Ar4, Ar5, Ar6, Ar7 and Ar8 are same or different, and are each independently selected from any one of a substituted or unsubstituted alkyl group, a substituted or unsubstituted aryl group, a substituted or unsubstituted heteroaryl group, a substituted of unsubstituted fused ring aryl group and a substituted or unsubstituted fused ring heteroaryl group.

In some embodiments, A1 and A2 are the same or different, and are each independently selected from any one of a substituted or unsubstituted phenyl group, a substituted or unsubstituted naphthyl group, a substituted or unsubstituted phenanthrenyl group, a substituted or unsubstituted benzofuranyl group, a substituted or unsubstituted benzothienyl group, a substituted or unsubstituted dibenzofuranyl group dibenzofuran and a substituted or unsubstituted dibenzothienyl group. B1 and B2 are the same or different, and are each independently selected from any one of a substituted or unsubstituted methylene group, a substituted or unsubstituted adamantyl group, and a substituted or unsubstituted cyclohexyl group.

In some embodiments, B1 and B2 are capable of bonding to be a ring.

In some embodiments, the first type of compound is a hole-type material used for transporting holes; and the second type of compound is an electron-type material used for transporting electrons.

In another aspect, a light-emitting device is provided. The light-emitting device includes at least two light-emitting units. Each light-emitting unit in the at least two light-emitting units includes a light-emitting layer, a first type of functional layer disposed on a side of the light-emitting layer, and a second type of functional layer disposed on another side of the light-emitting layer.

The first type of functional layer includes a plurality of hole transport functional layers, and at least two hole transport functional layers in the plurality of hole transport functional layers each include the first type of compound as described in any of the above embodiments. The second type of functional layer includes an electron transport functional layer, and the electron transport functional layer includes a second type of compound. Alternatively, the second type of functional layer includes a plurality of electron transport functional layers, and at least two electron transport functional layers in the plurality of electron transport functional layers each include the second type of compound.

The second type of compound is selected from any one of structures shown in a following general formula (II).

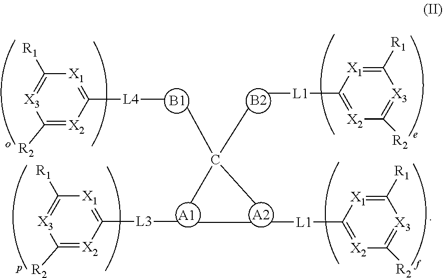

Where values of e, f, o and p are each independently selected from any one of 0, 1, 2, and 4, and at least one of e, f, o and p is not 0.

X₁, X₂ and X₃ are same or different, and are each independently selected from any one of —CR₃ and N, and at least one of X₁, X₂ and X₃ is N.

R₁, R₂ and R₃ are same or different, and are each independently selected from any one of a substituted or unsubstituted aryl group and a substituted or unsubstituted heteroaryl group.

In some embodiments, the light-emitting device further includes a charge generation unit disposed between two adjacent light-emitting units in the at least two light-emitting units. The charge generation unit includes a hole generation layer and an electron generation layer. The hole generation layer includes two materials, and at least one of the two materials is the first type of compound.

In some embodiments, the electron generation layer includes a fourth host material, and the fourth host material is selected from any one of structures shown in following general formula (III).

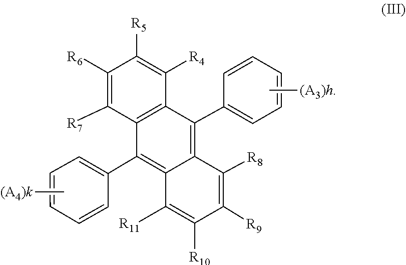

Where R₄, R₅, R₆, R₇, R₉, R₁₀, R₁₁, A₃ and A₄ are same or different, and are each independently selected from a phosphonoxy group, H, D, F, a substituted or unsubstituted C1 to C18 alkyl group, a substituted or unsubstituted C6 to C60 aryl group, or a substituted or unsubstituted C3 to C60 heteroaryl group. At least one of R₄, R₅, R₆, R₇, R₈, R₉, R₁₀, R₁₁, A₃ and A₄ is the phosphonoxy group. Values of k and h are each independently selected from any one of 0, 1, 2, 3, 4 and 5.

In some embodiments, two adjacent ones of R₄, R₅, R₆, R₇, R₈, R₉, R₁₀ and R₁₁ are capable of bonding to be a ring. In a case where h is greater than or equal to 2 (h≥2), adjacent A₃ are capable of bonding to be a ring. In a case where k is greater than or equal to 2 (k≥2), adjacent A₄ are capable of bonding to be a ring.

In some embodiments, the light-emitting layer includes a first sub-pixel film layer, a second sub-pixel film layer and a third sub-pixel film layer. The first sub-pixel film layer, the second sub-pixel film layer and the third sub-pixel film layer are arranged in a first direction. The first sub-pixel film layer is configured to emit one of red light, blue light and green light, the second sub-pixel film layer is configured to emit another of red light, blue light and green light, and the third sub-pixel film layer is configured to emit a last one of red light, blue light and green light.

In some embodiments, the first type of functional layer includes a hole transport layer and a plurality of electron blocking layers disposed between the hole transport layer and the light-emitting layer. At least two of the hole transport layer and the plurality of electron blocking layers each contain the first type of compound. The second type of functional layer includes the electron transport functional layer, the electron transport functional layer is a hole blocking layer, and the hole blocking layer contains the second type of compound. Alternatively, the second type of functional layer includes the plurality of electron transport functional layers, the plurality of electron transport functional layers include a hole blocking layer and an electron transport layer, and the hole blocking layer and the electron transport layer each contain the second type of compound.

In some embodiments, the plurality of electron blocking layers include a first electron blocking layer, a second electron blocking layer and a third electron blocking layer; the first electron blocking layer, the second electron blocking layer and the third electron blocking layer are arranged in the first direction. Alternatively, the plurality of electron blocking layers include a first electron blocking layer and a second electron blocking layer; the second electron blocking layer is disposed between the light-emitting layer and the hole transport layer; and the first electron blocking layer is disposed between the second electron blocking layer and the first sub-pixel film layer.

In some embodiments, the first sub-pixel film layer is configured to emit red light, and the first electron blocking layer is disposed between the first sub-pixel film layer and the hole transport layer. The first electron blocking layer and the hole transport layer each contain the first type of compound.

In some embodiments, the first sub-pixel film layer is configured to emit red light, and the first electron blocking layer is disposed between the first sub-pixel film layer and the hole transport layer. The second sub-pixel film layer is configured to emit green light, and the second electron blocking layer is disposed between the second sub-pixel film layer and the hole transport layer. The first electron blocking layer and the second electron blocking layer each contain the first type of compound.

In some embodiments, the plurality of electron blocking layers include a first electron blocking layer, a second electron blocking layer and a third electron blocking layer. The first sub-pixel film layer is configured to emit red light, and the first electron blocking layer is disposed between the first sub-pixel film layer and the hole transport layer. The second sub-pixel film layer is configured to emit green light, and the second electron blocking layer is disposed between the second sub-pixel film layer and the hole transport layer. The third sub-pixel film layer is configured to emit blue light, and the third electron blocking layer is disposed between the third sub-pixel film layer and the hole transport layer. A specific surface area of the first electron blocking layer is smaller than a specific surface area of the second electron blocking layer, and the specific surface area of the first electron blocking layer is smaller than a specific surface area of the third electron blocking layer.

In some embodiments, a turn-on voltage of the third sub-pixel film layer is greater than a turn-on voltage of the second sub-pixel film layer, and the turn-on voltage of the second sub-pixel film layer is greater than a turn-on voltage of the first sub-pixel film layer.

In some embodiments, the at least two light-emitting units include a first light-emitting unit and a second light-emitting unit. The first light-emitting unit, the charge generation unit and the second light-emitting unit are stacked in sequence in a second direction; the second direction is perpendicular to the first direction. The first type of functional layer, the light-emitting layer and the second type of functional layer of the first light-emitting unit are stacked in the second direction. The first type of functional layer of the first light-emitting unit further includes a hole injection layer, and the hole injection layer is disposed on a side of the hole transport layer away from the light-emitting layer.

The first type of functional layer, the light-emitting layer and the second type of functional layer of the second light-emitting unit are stacked in the second direction. The second type of functional layer of the second light-emitting unit includes the hole blocking layer and the electron transport layer, the second type of functional layer of the second light-emitting unit further includes an electron injection layer, and the electron injection layer is disposed on a side of the electron transport layer away from the light-emitting layer.

In some embodiments, a sub-pixel film layer of the first light-emitting unit and a sub-pixel film layer of the second light-emitting unit emit light of same color. A difference between a wavelength of light emitted by the sub-pixel film layer of the first light-emitting unit and a wavelength of light emitted by the sub-pixel film layer of the second light-emitting unit is less than or equal to 20 nm. The sub-pixel film layer of the first light-emitting unit and the sub-pixel film layer of the second light-emitting unit each include any one of the first sub-pixel film layer, the second sub-pixel film layer and the third sub-pixel film layer.

In some embodiments, a ratio of mobility of the hole blocking layer of the first light-emitting unit to mobility of the hole blocking layer of the second light-emitting unit is less than or equal to 10 and greater than or equal to 0.1; and a ratio of mobility of the hole transport layer of the first light-emitting unit to mobility of the hole transport layer of the second light-emitting unit is less than or equal to 10 and greater than or equal to 0.1.

In some embodiments, the light-emitting device further includes a charge generation unit disposed between the first light-emitting unit and the second light-emitting unit, and the charge generation unit includes a hole generation layer and an electron generation layer. A difference between a HOMO energy level of the hole generation layer and a HOMO energy level of the hole transport layer of the second light-emitting unit is less than or equal to 0.3 eV; and a difference between a LUMO energy level of the electron generation layer and a LUMO energy level of the hole blocking layer of the first light-emitting unit is less than or equal to 0.5 eV.

In some embodiments, a dipole moment of the electron generation layer is greater than 4 D.

In some embodiments, the electron generation layer further includes a fourth doping material, and the fourth doping material includes any one of alkali metals and oxides thereof, alkaline-earth metals and oxides thereof, and transition metals and oxides thereof.

In some embodiments, the first sub-pixel film layer includes at least one first host material and a first doping material, the at least one first host material includes two first host materials, and each of the two first host materials is any one of exciplexes, isomers and homologues.

The second sub-pixel film layer includes at least two second host materials and a second doping material, the at least two second host materials include two second host materials, and each of the two second host materials is any one of exciplexes, isomers and homologues.

The third sub-pixel film layer includes at least one third host material and a third doping material, the at least one third host material includes two third host materials, and each of the two third host materials is any one of exciplexes, isomers and homologues. A third host material in the at least one third host material contains an anthracene derivative.

In some embodiments, the light-emitting device further includes a first electrode and a second electrode, and the at least two light-emitting units and the charge generation unit are disposed between the first electrode and the second electrode.

In another aspect, a light-emitting substrate is provided. The light-emitting substrate includes the light-emitting device as described in any of the above embodiments.

In yet another aspect, a light-emitting apparatus is provided. The light-emitting apparatus includes the light-emitting substrate as described above.

### BRIEF DESCRIPTION OF THE DRAWINGS

In order to describe technical solutions in the present disclosure more clearly, accompanying drawings to be used in some embodiments of the present disclosure will be introduced briefly below. Obviously, the accompanying drawings to be described below are merely accompanying drawings of some embodiments of the present disclosure, and a person of ordinary skill in the art may obtain other drawings according to these drawings. In addition, the accompanying drawings to be described below may be regarded as schematic diagrams, but are not limitations on an actual size of a product, an actual process of a method and an actual timing of a signal to which the embodiments of the present disclosure relate.

FIG. 1 is a structural diagram of a light-emitting device, in accordance with some embodiments of the present disclosure;

FIG. 2 is a structural diagram of another light-emitting device, in accordance with some embodiments of the present disclosure;

FIG. 3 is a structural diagram of yet another light-emitting device, in accordance with some embodiments of the present disclosure;

FIG. 4 is a flow diagram of a method for manufacturing a light-emitting device, in accordance with some embodiments of the present disclosure;

FIG. 5 is a flow diagram of part of a method for manufacturing a light-emitting device, in accordance with some embodiments of the present disclosure;

FIG. 6 is a structural diagram of a light-emitting substrate, in accordance with some embodiments of the present disclosure; and

FIG. 7 is a structural diagram of a light-emitting apparatus, in accordance with some embodiments of the present disclosure.

### DETAILED DESCRIPTION

Technical solutions in some embodiments of the present disclosure will be described clearly and completely with reference to the accompanying drawings below. Obviously, the described embodiments are merely some but not all embodiments of the present disclosure. All other embodiments obtained by a person of ordinary skill in the art based on the embodiments of the present disclosure shall be included in the protection scope of the present disclosure.

Unless the context requires otherwise, throughout the description and the claims, the term “comprise” and other forms thereof such as the third-person singular form “comprises” and the present participle form “comprising” are construed as open and inclusive, i.e., “including, but not limited to”, In the description of the specification, the terms such as “one embodiment”, “some embodiments”, “exemplary embodiments”, “example”, “specific example” or “some examples” are intended to indicate that specific features, structures, materials or characteristics related to the embodiment(s) or example(s) are included in at least one embodiment or example of the present disclosure. Schematic representations of the above terms do not necessarily refer to the same embodiment(s) or example(s). In addition, the specific features, structures, materials, or characteristics described herein may be included in any one or more embodiments or examples in any suitable manner.

Hereinafter, the terms such as “first” and “second” are used for descriptive purposes only, and are not to be construed as indicating or implying the relative importance or implicitly indicating the number of indicated technical features. Thus, features defined with “first” or “second” may explicitly or implicitly include one or more of the features. In the description of the embodiments of the present disclosure, the term “a plurality of” or “the plurality of” means two or more unless otherwise specified.

The phrase “at least one of A, B and C” has a same meaning as the phrase “at least one of A, B or C”, and they both include the following combinations of A, B and C: only A, only B, only C, a combination of A and B, a combination of A and C, a combination of B and C, and a combination of A, B and C.

The phrase “A and/or B” includes the following three combinations: only A, only B, and a combination of A and B.

It will be understood that when a layer or element is referred to as being on another layer or substrate, the layer or element may be directly on the another layer or substrate, or there may be intermediate layer(s) between the layer or element and the another layer or substrate.

Exemplary embodiments are described herein with reference to sectional views and/or plan views as idealized exemplary drawings. In the accompanying drawings, thicknesses of layers and sizes of regions are enlarged for clarity. Variations in shapes relative to the accompanying drawings due to, for example, manufacturing technologies and/or tolerances may be envisaged. Therefore, the exemplary embodiments should not be construed to be limited to the shapes of regions shown herein, but to include deviations in the shapes due to, for example, manufacturing. For example, an etched region shown in a rectangular shape generally has a feature of being curved. Therefore, the regions shown in the accompanying drawings are schematic in nature, and their shapes are not intended to show actual shapes of the regions in an apparatus, and are not intended to limit the scope of the exemplary embodiments.

In the field of organic semiconductors, an organic light-emitting diode (OLED) technology has been successfully used in commercial flat-panel display and lighting industries. The OLED has characteristics of self-luminescence, no need for a backlight source, and a small thickness and a light weight of the panel. In addition, the OLED also has advantages of wide viewing angle, large contrast, fast response time, wide operating temperature range and flexibility. Stacked OLED devices play an important role in the field of OLED display and lighting.

First of all, the OLED emits light driven by current. When driven by the current of the same density, luminous brightness of a stacked OLED composed of n identical light-emitting units is n times luminous brightness of a traditional OLED composed of a single light-emitting unit. Therefore, the current efficiency of the stacked OLED is n times the current efficiency of the traditional OLED. However, the driving voltage of the stacked OLED is also n times the driving voltage of the traditional OLED. Therefore, the power and efficiency of the stacked OLED is not significantly improved.

Secondly, OLED display and lighting equipment works at a certain brightness. Under the same luminous brightness, the current density for driving the stacked OLED is 1/n of the current density for driving the traditional OLED. The greater the current density of the OLED, the faster the aging of the OLED, and the shorter the life of the light-emitting device. Therefore, the life of the stacked OLED will be prolonged.

Furthermore, with the development of organic light-emitting devices, compositions of various compounds applied to various organic material layers are different, which may produce a large difference in the overall performance of the organic light-emitting device.

Since the stacked OLED is composed of a plurality of light-emitting units connected by charge generation layer(s) in a direction perpendicular to a light-emitting surface, the structure of the charge generation layer(s) in the stacked OLED plays a role of connecting all the OLED units. Moreover, three processes of efficient charge generation, fast charge transport and effective injection are indispensable in the stacked OLED, all of which have a significant impact on the performance of the light-emitting device.

Therefore, a reasonable combination of the light-emitting units in the stacked OLED and the charge generation layer(s) each between light-emitting units may ensure the efficient generation, injection and transport of charges.

In the related art, OLED devices generally use hole transport (HT) type materials and P-type dopants for co-evaporation to obtain a hole transport layer. The lateral resistance of this type of materials is small, especially after P-type doping, the resistance further decreases. Moreover, for sub-pixels of different colors, such as red sub-pixels, green sub-pixels and blue sub-pixels, turn-on voltages thereof have the following relationship: blue>green>red. Therefore, under low gray scale, when a single sub-pixel is lit up, there is a color crosstalk phenomenon that adjacent sub-pixels are also lit up, resulting in a poor color purity, a serious color mixing effect, and a poor display effect of the OLED. For example, when the green sub-pixel works, charges flows laterally to the red sub-pixel, and the red color sub-pixel is lit up, resulting in color crosstalk.

In light of this, the embodiments of the present disclosure provide a functional layer material. The functional layer material includes a compound centered on an sp3 hybridized carbon atom.

For example, the compound centered on the sp3 hybridized carbon atom (C) has characteristics shown in the following structural formula

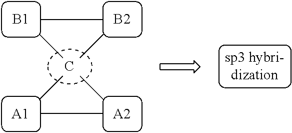

It will be noted that sp3 hybridization refers to a process of hybridization of one ns orbital and three np orbitals in the same electron shell of an atom. After sp3 hybridization of the atom, the ns orbital and the np orbitals above will be converted into four equivalent atomic orbitals, called “sp3 hybrid orbitals”. The symmetry axes of the four sp3 hybrid orbitals have same angles therebetween, which are all 109° 28′.

Taking the above carbon atom (C) as an example, the paired 2s electrons of the carbon atom (C) are separated, and one of the electrons runs into the 2p orbital with a slightly high energy. This process is called electronic transition. Then, hybridization is carried out, one 2s orbital and three 2p orbitals are hybridized to generate four sp3 hybrid orbitals with equal energies. Due to average mixture, each sp3 hybrid orbital contains 1/4 of s orbital component and 3/4 of p orbital component, each of which has 1 single electron. Then, these four electrons are paired with electrons on four groups (e.g., A1, A2, B1 and B2) to form bonds, thereby forming the above compound centered on the sp3 hybridized carbon atom (C).

In some embodiments, the compound centered on the sp3 hybridized carbon atom include a first type of compound, and the first type of compound is selected from any of structures shown in the following general formula I).

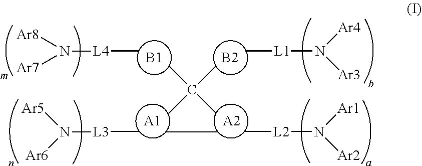

Where values of a, b, m and n are each independently selected from any of 0, 1, 2, 3 and 4, and at least one of a, b, m and n is not 0. A1 and A2 are the same or different, and are each independently selected from any of a substituted or unsubstituted trivalent aryl group, a substituted or unsubstituted fused ring trivalent aryl group, and a substituted or unsubstituted fused ring trivalent heteroaryl group. B1 and B2 are the same or different, and are each independently selected from any of a substituted or unsubstituted alkylene group, a substituted or unsubstituted arylene group, and a substituted or unsubstituted heteroarylene group.

L1, L2, L3 and L4 are the same or different, and are each independently selected from any of a single bond, a substituted or unsubstituted phenylene group, a substituted or unsubstituted biphenylene group, a substituted or unsubstituted terphenylene group, a substituted or unsubstituted fluorenyl group, a substituted or unsubstituted adamantyl group, and a substituted or unsubstituted heteroarylene group. Ar1, Ar2, Ar3, Ar4, Ar5, Ar6, Ar7 and Ar8 are the same or different, and are each independently selected from any of a substituted or unsubstituted alkyl group, a substituted or unsubstituted aryl group, a substituted or unsubstituted heteroaryl group, a substituted of unsubstituted fused ring aryl group and a substituted or unsubstituted fused ring heteroaryl group.

It will be noted that the aryl group may be a phenyl group; the heteroaryl group may be a furyl group, a pyranyl group, a thienyl group, a pyridyl group, or the like; the fused ring aryl group may be a naphthyl group, a phenanthrenyl group, or the like; and the fused ring heteroaryl group may be a benzofuranyl group, a benzothienyl group, a dibenzofuranyl group, a dibenzothienyl group, or the like. Moreover, the phenyl group refers to a general name of a group left after a hydrogen atom of one carbon atom on the benzene ring is removed; the phenylene group refers to a general name of a group left after hydrogen atoms of two carbon atoms on the benzene ring are removed; and a trivalent phenyl group refers to a general name of a group left after hydrogen atoms of three carbon atoms on the benzene ring are removed. For understandings of other terms such as the trivalent aryl group, the fused ring trivalent aryl group and the fused ring trivalent heteroaryl group, reference may be made to the above contents, and details are not repeated here. That is, the trivalent aryl group refers to a general name of a group left after hydrogen atoms of three carbon atoms on an aromatic hydrocarbon are removed; the fused ring trivalent aryl group refers to a general name of a group left after hydrogen atoms of three carbon atoms on a fused ring aromatic hydrocarbon are removed; and the fused ring trivalent heteroaryl group refers to a general name of a group left after hydrogen atoms of three carbon atoms on a fused ring aromatic hydrocarbon with hetero atoms are removed.

The values of a, b, m and n are each independently represent the number of a respective group.

For example, A1 and A2 are the same or different, and are each independently selected from any of a substituted or unsubstituted phenyl group, a substituted or unsubstituted naphthyl group, a substituted or unsubstituted phenanthrenyl group, a substituted or unsubstituted benzofuranyl group, a substituted or unsubstituted benzothienyl group, a substituted or unsubstituted dibenzofuranyl group dibenzofuran and a substituted or unsubstituted dibenzothienyl group. B1 and B2 are the same or different, and are each independently selected from any of a substituted or unsubstituted methylene group, a substituted or unsubstituted adamantyl group, and a substituted or unsubstituted cyclohexyl group.

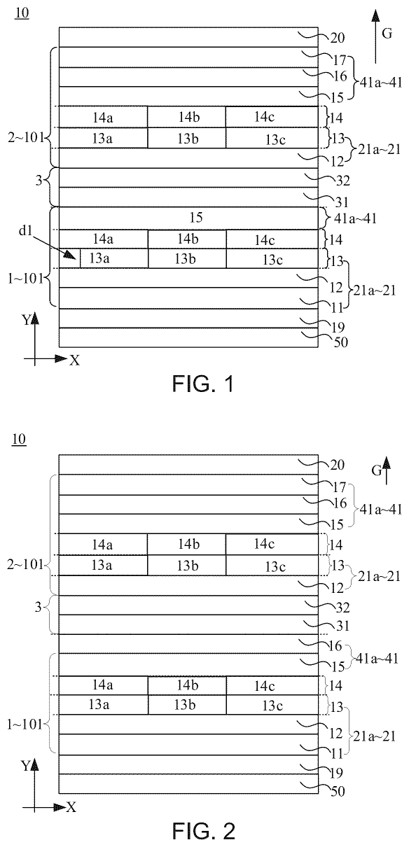

By providing the groups of A1, A2, B1 and B2, the first type of compound centered on the sp3 hybridized carbon atom with a stable structure is formed. By providing the N-containing groups connected to L1, L2, L3 and L4, the first type of compound is a hole-type material and may be used to transport holes. For example, as shown in FIG. 1, in the structural diagram of the light-emitting device 10, the first type of compound is used to form film layer(s), such as a first electron blocking layer 13a, in a hole transport functional layer 21a. The hole transport functional layer 21a is used to transport holes to a light-emitting layer 14 (for an introduction to the structure of the light-emitting device 10, reference is made to the following contents, and details are not provided here).

Exemplary structures of the first type of compound in the structure represented by the general formula (I) are introduced below.

In some examples, in a case where the values of a, m and n are 0, the value of b is 1, and L1 is selected as a single bond, the structural formula of the first type of compound may be as shown in the following formulas.

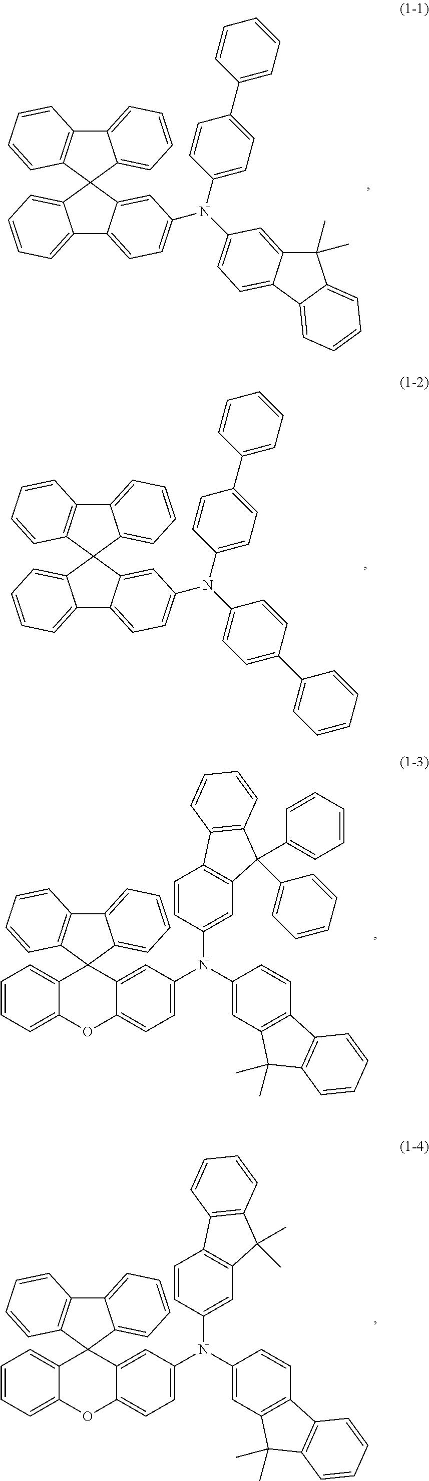 [SMILES: CC1(C)c2ccccc2-c2ccc(N(c3ccc(-c4ccccc4)cc3)c3ccc4c(c3)C3(c5ccccc5-c5ccccc53)c3ccccc3-4)cc21 ; c1ccc(-c2ccc(N(c3ccc(-c4ccccc4)cc3)c3ccc4c(c3)C3(c5ccccc5-c5ccccc53)c3ccccc3-4)cc2)cc1 ; CC1(C)c2ccccc2-c2ccc(N(c3ccc4c(c3)C3(c5ccccc5O4)c4ccccc4-c4ccccc43)c3ccc4c(c3)C(c3ccccc3)(c3ccccc3)c3ccccc3-4)cc21 ; CC1(C)c2ccccc2-c2ccc(N(c3ccc4c(c3)C(C)(C)c3ccccc3-4)c3ccc4c(c3)C3(c5ccccc5O4)c4ccccc4-c4ccccc43)cc21]

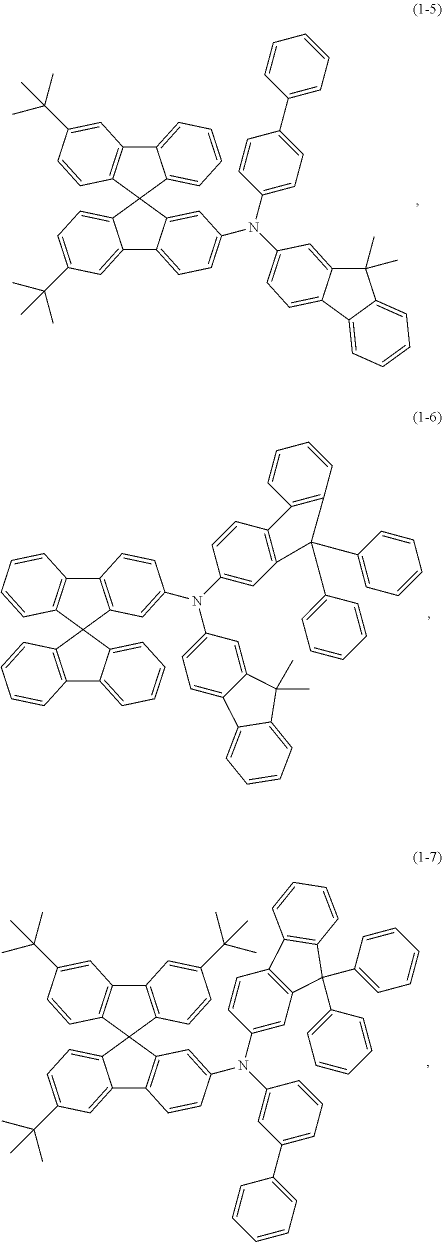 [SMILES: CC(C)(C)c1ccc2c(c1)-c1ccccc1C21c2ccc(C(C)(C)C)cc2-c2ccc(N(c3ccc(-c4ccccc4)cc3)c3ccc4c(c3)C(C)(C)c3ccccc3-4)cc21 ; CC1(C)c2ccccc2-c2ccc(N(c3ccc4c(c3)C(c3ccccc3)(c3ccccc3)c3ccccc3-4)c3ccc4c(c3)C3(c5ccccc5-c5ccccc53)c3ccccc3-4)cc21 ; CC(C)(C)c1ccc2c(c1)-c1cc(C(C)(C)C)ccc1C21c2ccc(C(C)(C)C)cc2-c2ccc(N(c3cccc(-c4ccccc4)c3)c3ccc4c(c3)C(c3ccccc3)(c3ccccc3)c3ccccc3-4)cc21]

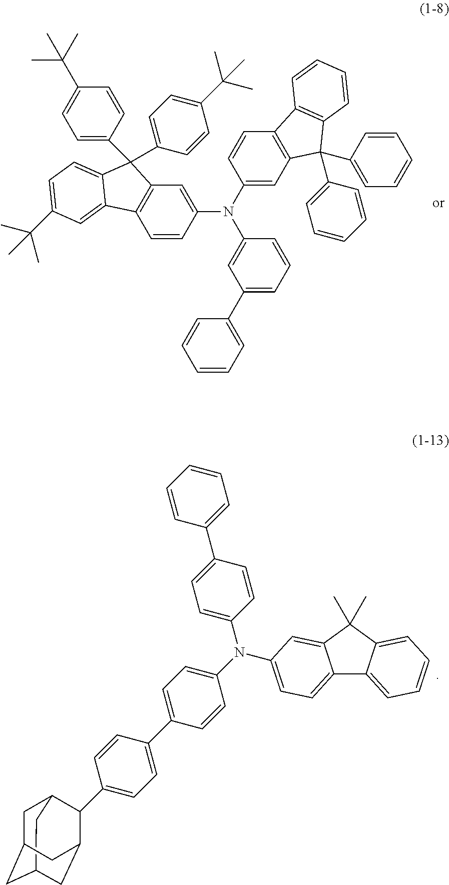 [SMILES: CC(C)(C)c1ccc(C2(c3ccc(C(C)(C)C)cc3)c3ccc(C(C)(C)C)cc3-c3ccc(N(c4cccc(-c5ccccc5)c4)c4ccc5c(c4)C(c4ccccc4)(c4ccccc4)c4ccccc4-5)cc32)cc1 ; CC1(C)c2ccccc2-c2ccc(N(c3ccc(-c4ccccc4)cc3)c3ccc(-c4ccc(C5C6CC7CC(C6)CC5C7)cc4)cc3)cc21]

It will be noted that (1-x) in the above structural formulas is a name of each structural formula but is not part of the structure in the structural formula, where x is a positive integer.

It can be seen from the first type of compounds shown in the above structural formulas (1-1) to (1-7) that, B1 and B2 may bond to form a ring. For example, in the first type of compound shown in the above structural formula (1-1), B1 and B2 bond to form a ring by a single bond connection. In the first type of compound shown in the above structural formula (1-4), B1 and B2 bond to form a ring by connecting an oxygen atom (O).

B1 and B2 bond to form a ring, so that the rigidity of the first type of compound may be improved, that is, the stability of the first type of compound may be improved. For example, when the first type of compound is used to form a film layer such as the first electron blocking layer 13a (as shown in FIG. 1) by evaporation, the structural stability of the material may be ensured, thereby effectively ensuring that the formed film layer has good performance.

In some examples, in a case where the values of b and m are 0, the values of a and n are 1, and L2 and L3 are selected as single bonds, the structural formula of the first type of compound may be as shown in the following formulas.

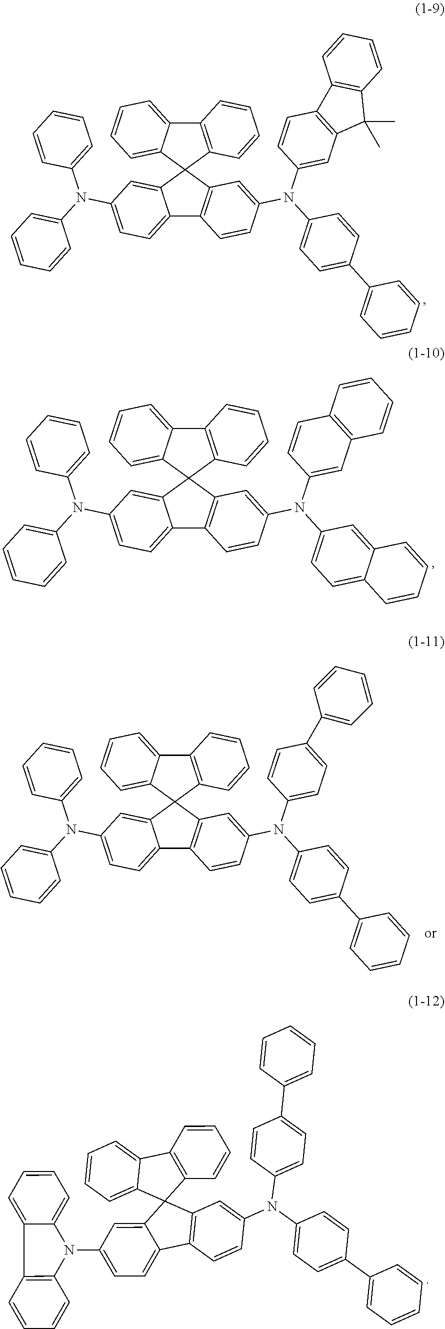

It will be noted that the structural formulas listed above are examples of the structure of the first type of compound but are not limitations on the structure of the first type of compound.

The structure of another compound centered on an sp3 hybridized carbon atom (C) will be introduced in the following.

In some embodiments, the compound centered on the sp3 hybridized carbon atom (C) include a second type of compound, and the second type of compound is selected from any of structures shown in the following general formula (II).

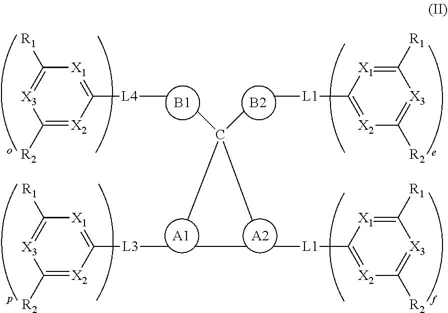

Where values of e, f, o and p are each independently selected from any of 0, 1, 2, 3 and 4, and at least one of e, f, o and p is not 0. X₁, X₂ and X₃ are the same or different, and are each independently selected from any of —CR₃ and N, and at least one of X₁, X₂ and X₃ is N. R₁, R₂ and R₃ are the same or different, and are each independently selected from a substituted or unsubstituted aryl group and a substituted or unsubstituted heteroaryl group.

It will be noted that for the structures of A1, A2, L1, L2, L3 and L4 in the second type of compound, reference may be made to the introduction of the structures of A1, A2, L1, L2, L3 and L4 in the first type of compound.

That is, A1 and A2 are the same or different, and are each independently selected from any of a substituted or unsubstituted trivalent aryl group, a substituted or unsubstituted fused ring trivalent aryl group, and a substituted or unsubstituted fused ring trivalent heteroaryl group. B1 and B2 are the same or different, and are each independently selected from any of a substituted or unsubstituted alkylene group, a substituted or unsubstituted arylene group, and a substituted or unsubstituted heteroarylene group.

L1, L2, L3 and L4 are the same or different, and are each independently selected from any of a single bond, a substituted or unsubstituted phenylene group, a substituted or unsubstituted biphenylene group, a substituted or unsubstituted terphenylene group, a substituted or unsubstituted fluorenyl group, a substituted or unsubstituted adamantyl group, and a substituted or unsubstituted heteroarylene group. Ar1, Ar2, Ar3, Ar4, Ar5, Ar6, Ar7 and Ar8 are the same or different, and are each independently selected from any of a substituted or unsubstituted alkyl group, a substituted or unsubstituted aryl group, a substituted or unsubstituted heteroaryl group, a substituted of unsubstituted fused ring aryl group and a substituted or unsubstituted fused ring heteroaryl group.

By providing the N-containing cyclic groups connected to L1, L2, L3 and L4, the second type of compound is an electron-type material for transporting electrons. For example, as shown in FIG. 1, in the structural diagram of the light-emitting device 10, the second type of compound is used to form film layer(s), such as a hole blocking layer 15, in an electron transport functional layer 41a. The electron transport functional layer 41a is used to transport electrons to the light-emitting layer 14 (for an introduction to the structure of the light-emitting device 10, reference is made to the following contents, and details are not provided here).

Exemplary structures of the second type of compound in the structure represented by the general formula (II) are introduced below.

In some examples, in a case where the values of e, f and o are 0, the value of p is 1, and L3 is selected as a biphenylene group, the structural formula of the second type of compound may be as shown in the following formulas.

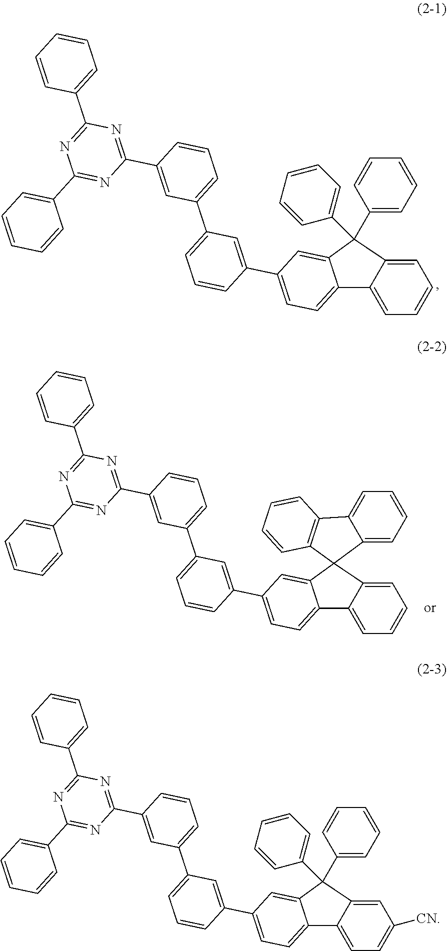 [SMILES: c1ccc(-c2nc(-c3ccccc3)nc(-c3cccc(-c4cccc(-c5ccc6c(c5)C(c5ccccc5)(c5ccccc5)c5ccccc5-6)c4)c3)n2)cc1 ; c1ccc(-c2nc(-c3ccccc3)nc(-c3cccc(-c4cccc(-c5ccc6c(c5)C5(c7ccccc7-c7ccccc75)c5ccccc5-6)c4)c3)n2)cc1 ; N#Cc1ccc2c(c1)C(c1ccccc1)(c1ccccc1)c1cc(-c3cccc(-c4cccc(-c5nc(-c6ccccc6)nc(-c6ccccc6)n5)c4)c3)ccc1-2]

In some examples, in a case where the values of e, f and o are 0, the value of p is 1, and L3 is selected as a dibenzofuranyl group, the structural formula of the second type of compound may be as shown in the following formulas.

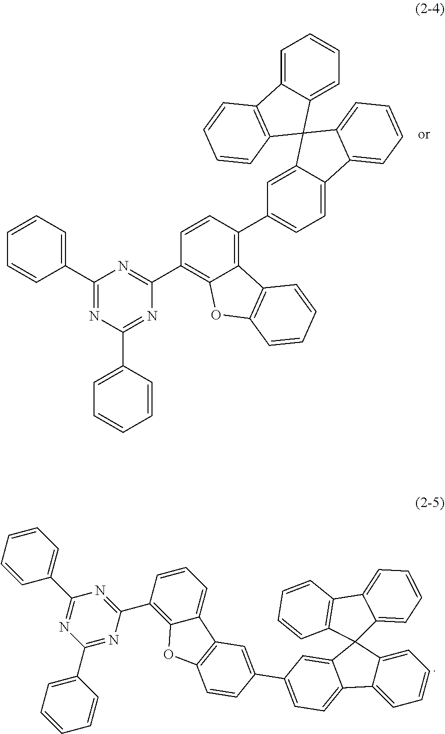 [SMILES: c1ccc(-c2nc(-c3ccccc3)nc(-c3ccc(-c4ccc5c(c4)C4(c6ccccc6-c6ccccc64)c4ccccc4-5)c4c3oc3ccccc34)n2)cc1 ; c1ccc(-c2nc(-c3ccccc3)nc(-c3cccc4c3oc3ccc(-c5ccc6c(c5)C5(c7ccccc7-c7ccccc75)c5ccccc5-6)cc34)n2)cc1]

In some examples, in a case where the values of e and o are 0, the values of f and p are 1, and L2 and L3 are selected as single bonds, the structural formula of the second type of compound may be as shown in the following formula.

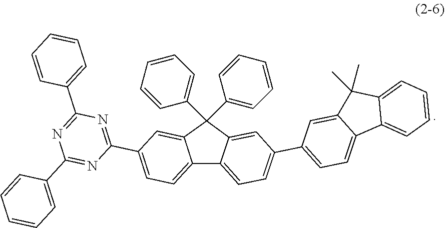 [SMILES: CC1(C)c2ccccc2-c2ccc(-c3ccc4c(c3)C(c3ccccc3)(c3ccccc3)c3cc(-c5nc(-c6ccccc6)nc(-c6ccccc6)n5)ccc3-4)cc21]

In some examples, in a case where the values of e, f and o are 0, the value of p is 1, and L3 is selected as a single bond, the structural formula of the second type of compound may be as shown in the following formulas.

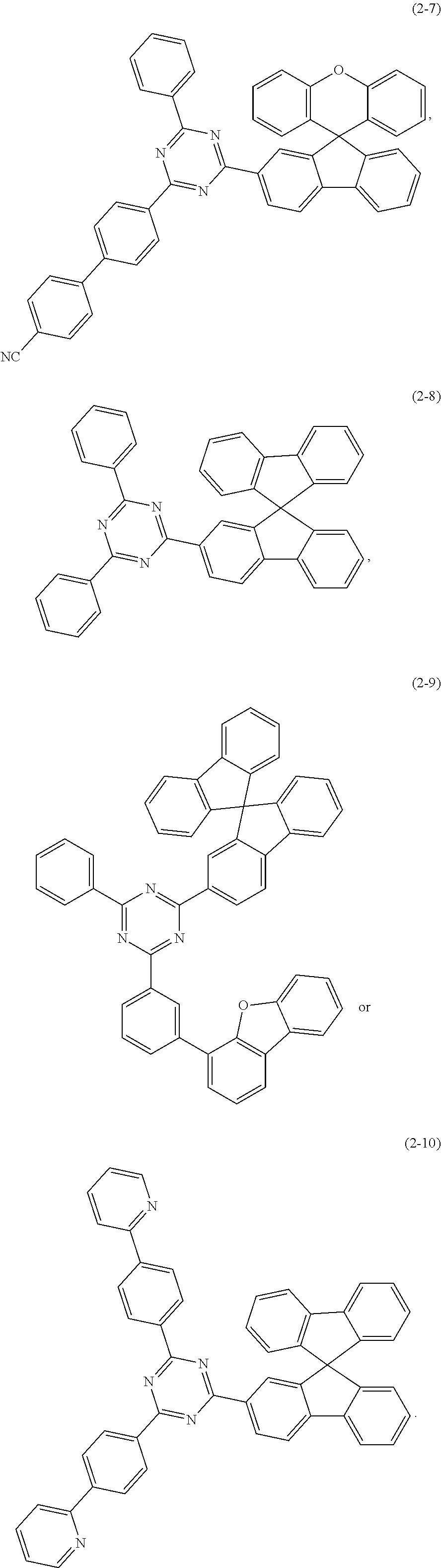 [SMILES: N#Cc1ccc(-c2ccc(-c3nc(-c4ccccc4)nc(-c4ccc5c(c4)C4(c6ccccc6Oc6ccccc64)c4ccccc4-5)n3)cc2)cc1 ; c1ccc(-c2nc(-c3ccccc3)nc(-c3ccc4c(c3)C3(c5ccccc5-c5ccccc53)c3ccccc3-4)n2)cc1 ; c1ccc(-c2nc(-c3cccc(-c4cccc5c4oc4ccccc45)c3)nc(-c3ccc4c(c3)C3(c5ccccc5-c5ccccc53)c3ccccc3-4)n2)cc1 ; c1ccc(-c2ccc(-c3nc(-c4ccc(-c5ccccn5)cc4)nc(-c4ccc5c(c4)C4(c6ccccc6-c6ccccc64)c4ccccc4-5)n3)cc2)nc1]

In some examples, in a case where the values of e, f and o are 0, the value of p is 1, and L3 is selected as a phenylene group or a naphthylene group, the structural formula of the second type of compound may be as shown in the following formulas.

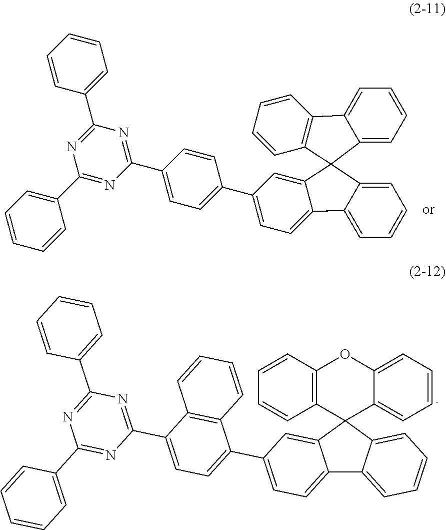 [SMILES: c1ccc(-c2nc(-c3ccccc3)nc(-c3ccc(-c4ccc5c(c4)C4(c6ccccc6-c6ccccc64)c4ccccc4-5)cc3)n2)cc1 ; c1ccc(-c2nc(-c3ccccc3)nc(-c3ccc(-c4ccc5c(c4)C4(c6ccccc6Oc6ccccc64)c4ccccc4-5)c4ccccc34)n2)cc1]

Similarly, (2-x) in the above structural formulas is a name of each structural formula but is not part of the structure in the structural formula, where x is a positive integer.

It can be seen from the second type of compounds shown in the above structural formulas (2-2), (2-4), (2-5), (2-7), (2-8), (2-9), (2-10), (2-11) and (2-12) that B1 and B2 may bond to form a ring. For example, in the second type of compound shown in the above structural formula (2-2), B1 and B2 bond to form a ring by a single bond connection. In the second type of compound shown in the above structural formula (2-7), B1 and B2 bond to form a ring by connecting an oxygen atom (O).

Similarly, B1 and B2 bond to form a ring, so that the rigidity of the second type of compound may be improved, that is, the stability of the second type of compound may be improved. For example, when the second type of compound is used to form a film layer such as the hole blocking layer 15 (as shown in FIG. 1) by evaporation, the structural stability of the material may be ensured, thereby effectively ensuring that the formed film layer has good performance.

It will be noted that the structural formulas listed above are examples of the structure of the second type of compound but are not limitation on the structure of the second type of compound.

In another aspect, as shown in FIGS. 1 to 3, a light-emitting device 10 is provided, and the structure of the light-emitting device 10 is introduced below.

In some embodiments, as shown in FIGS. 1 to 3, the light-emitting device 10 includes at least two light-emitting units 101, and each light-emitting unit 101 in the at least two light-emitting units 101 includes a light-emitting layer 14, a first type of functional layer 21 disposed on a side of the light-emitting layer 14, and a second type of functional layer 41 disposed on another side of the light-emitting layer 14.

The first type of functional layer 21 includes a plurality of hole transport functional layers 21a, and at least two hole transport functional layers 21a in the plurality of hole transport functional layers 21a each include the first type of compound as described in any of the above embodiments.

The second type of functional layer 41 includes an electron transport functional layer 41a, and an electron transport functional layer 41a includes the second type of compound as described in any of the above embodiments. Alternatively, the second type of functional layer 41 includes a plurality of electron transport functional layers 41a, and at least two electron transport functional layer 41a in the plurality of electron transport functional layers 41a each include the second type of compound as described in any of the above embodiments.

In some examples, as shown in FIG. 1, the light-emitting device 10 includes two light-emitting units 101, and the two light-emitting units 101 are a first light-emitting unit 1 and a second light-emitting unit 2. The first light-emitting unit 1 and the second light-emitting unit 2 each include a light-emitting layer 14, a first type of functional layer 21 provided on a side of the light-emitting layer 14, and a second type of functional layer 41 provided on another side of the light-emitting layer 14. The first type of functional layer 21 is used to transport holes, and the second type of functional layer 41 is used to transport electrons.

The light-emitting device 10 is provided to include a plurality of light-emitting units 101 such as two light-emitting units 101, so as to form a stacked light-emitting device 10. Thus, the brightness of the light-emitting device 10 may be improved and the life of the light-emitting device 10 may be extended.

For example, as shown in FIG. 1, considering the first light-emitting unit 1 of the light-emitting device 10 as an example, the first type of functional layer 21 of the first light-emitting unit 1 includes a plurality of hole transport functional layers 21a. For example, the plurality of hole transport functional layers 21a are a hole injection layer 11, a hole transport layer 12 and an electron blocking layer 13, and the electron blocking layer 13 may include a first electron blocking layer 13a, a second electron blocking layer 13b and a third electron blocking layer 13c. In the plurality of hole transport functional layers 21a formed by the hole injection layer 11, the hole transport layer 12, the first electron blocking layer 13a, the second electron blocking layer 13b and the third electron blocking layer 13c, at least two holes transport functional layers 21a each include the first type of compound as described in any of the above embodiments.

For example, as shown in FIG. 1, considering the second light-emitting unit 2 of the light-emitting device 10 as an example, the first type of functional layer 21 of the second light-emitting unit 2 includes a plurality of hole transport functional layers 21a. For example, the plurality of hole transport functional layers 21a include a hole transport layer 12 and an electron blocking layer 13, and the electron blocking layer 13 may include a first electron blocking layer 13a, a second electron blocking layer 13b and a third electron blocking layer 13c. In the plurality of hole transport functional layers 21a formed by the hole transport layer 12, the first electron blocking layer 13a, the second electron blocking layer 13b and the third electron blocking layer 13c, at least two holes transport functional layers 21a each include the first type of compound as described in any of the above embodiments.

The first type of compound, as a hole transport type material, may achieve transport of holes. Moreover, the provision of the at least two hole transport functional layers 21a each including the first type of compound may adjust physical property match between the hole transport functional layers 21a, reduce a hole transport energy level difference between the plurality of hole transport functional layers 21a, thereby ensuring smooth transport of holes.

For example, as shown in FIG. 1, considering the first light-emitting unit 1 of the light-emitting device 10 as an example, the second type of functional layer 41 of the first light-emitting unit 1 includes an electron transport functional layer 41a. The electron transport functional layer 41a is a hole blocking layer 15, and the hole blocking layer 15 includes the second type of compound as described in any of the above embodiments. Considering the second light-emitting unit 2 of the light-emitting device 10 as an example, the second type of functional layer 41 of the second light-emitting unit 2 includes a plurality of electron transport functional layers 41a. For example, the plurality of electron transport functional layers 41a are a hole blocking layer 15, an electron transport layer 16 and an electron injection layer 17, and in the plurality of electron transport functional layers 41a formed by the hole blocking layer 15, the electron transport layer 16 and the electron injection layer 17, at least two electron transport functional layers 41a each include the second type of compound as described in any of the above embodiments.

The second type of compound, as an electron transport type material, may achieve transport of electrons. Moreover, the provision of the electron transport functional layer 41a including the second type of compound may reduce an electron transport energy level difference, thereby ensuring smooth transport of electrons.

By providing the first type of compound with the hole transport function in the hole transport functional layer 21a, the hole transport functional layer 21a may smoothly transport holes to the light-emitting layer 14. By providing the second type of compound with the electron transport function in the electron transport functional layer 41a, the electron transport functional layer 41a may smoothly transport electrons to the light-emitting layer 14.

Moreover, at least two hole transport functional layers 21a in the plurality of hole transport functional layers 21a each are provided to include the first type of compound, and at least two electron transport functional layers 41a in the plurality of electron transport functional layers 41a each are provided to or one electron transport functional layer 41a is provided to include the second type of compound, so that a transport energy level difference of charges (including electrons or holes) may be reduced. That is to say, a plurality of functional layers (including the hole transport functional layers 21a or the electron transport functional layers 41a) contain similar materials with sp3 hybridized structure, so that the transport energy level differences of holes or electrons between different functional layers may be reduced, thereby making injection and transport of electrons or holes easy.

The electrons and the holes achieve balance in the light-emitting layer 14, that is, the electrons and the holes combine in the light-emitting layer 10 to form excitons to further emit light, thereby improving the luminous efficiency of the light-emitting device 10 and reducing the turn-on voltage of the light-emitting device 10.

In some embodiments, as shown in FIGS. 1 to 3, the light-emitting device 10 further includes a charge generation unit 3 disposed between two adjacent light-emitting units 101 in the at least two light-emitting units 101. The charge generation unit 3 includes a hole generation layer 32 and an electron generation layer 31. The hole generation layer 32 includes two materials, and at least one of the two materials is a first type of compound.

In some examples, as shown in FIG. 1, the light-emitting device 10 includes a first light-emitting unit 1 and a second light-emitting unit 2. A charge generation unit 3 is provided between the first light-emitting unit 1 and the second light-emitting unit 2. The charge generation unit has a function of connecting the first light-emitting unit 1 and the second light-emitting unit 2; moreover, the provision of the charge generation unit 3 contributes to generation of charges (electrons or holes).

For example, the hole generation layer 32 is used to generate holes, and the holes generated by the hole generation layer 32 are transported to the light-emitting layer 14 of the second light-emitting unit 2 through the hole transport layer 12 of the second light-emitting unit 2. For example, a first type of compound centered on an sp3 hybridized carbon atom is provided in the hole generation layer 32, and a first type of compound centered on an sp3 hybridized carbon atom is provided in the hole transport layer 12 of the second light-emitting unit 2. In this way, it is beneficial to control of holes and balance of hole transport, so that holes may be smoothly injected into the light-emitting layer 14 of the second light-emitting unit 2.

For example, one material in the hole generation layer 32 is a first type of compound centered on an sp3 hybridized carbon atom, and another material in the hole generation layer 32 is any of N,N′-di(1-naphthyl)-N,N-diphenyl-(1,1′-biphenyl)-4,4′-diamine (NPB), hexaazatriphenylene-hexacarbonitrile (HATCN) and a triaxial alkali compound, and a structural formula of the another material in the hole generation layer 32 may be selected from any of the structural formulas shown below.

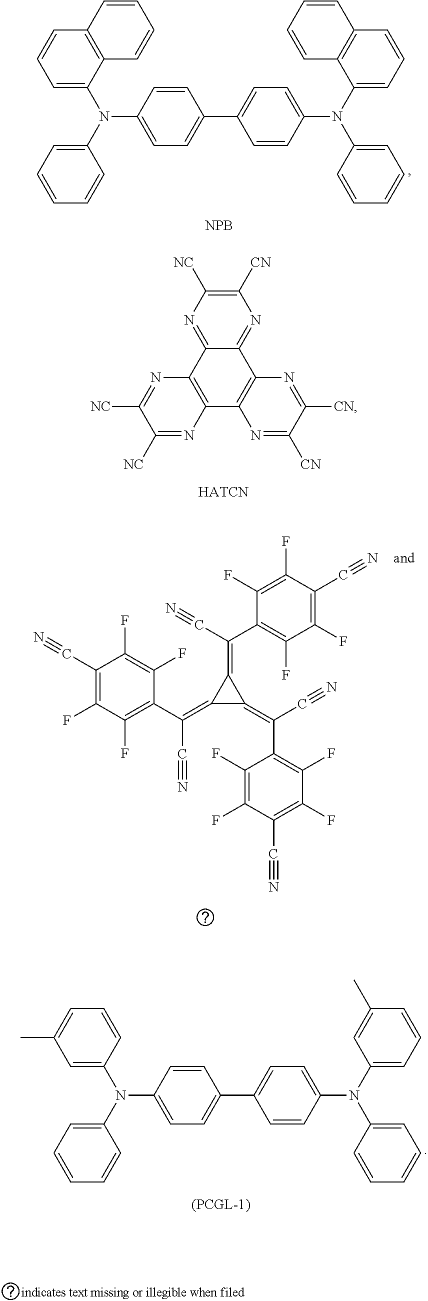

For example, as shown in FIG. 1, the electron generation layer 31 is used to generate electrons, and the electrons are injected into the light-emitting layer 14 of the first light-emitting unit 1 and combined with holes transported to the light-emitting layer 14 from the hole transport functional layer 21a to generate excitons.

The structure of the material of the electron generation layer 31 is described below.

In some embodiments, as shown in FIG. 1, the electron generation layer 31 includes a fourth host material, and the fourth host material is selected from any of structures shown in the following general formula (III).

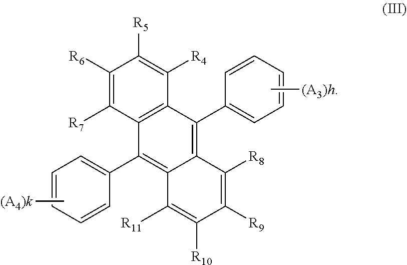

Where R₄, R₅, R₆, R₇, R₈, R₉, R₁₀, R₁₁, A₃ and A₄ are the same or different, and are each independently selected from a phosphonoxy group, H, D, F, a substituted or unsubstituted C1 to C18 alkyl group, a substituted or unsubstituted C6 to C60 aryl group, or a substituted or unsubstituted C3 to C60 heteroaryl group. In addition, at least one of R₄, R₅, R₆, R₇, R₈, R₉, R₁₀, R₁₁, A₃ and A₄ is the phosphonoxy group. The values of k and h are each independently selected from any of 0, 1, 2, 3, 4 and 5.

Using an anthracene derivative containing a phosphorus-oxygen bond as the host material of the electron generation layer 31 is beneficial to injection of the electrons, thereby improving the efficiency of the light-emitting device 10.

In some examples, the structural formula of the fourth host material of the electron generation layer 31 may be as shown in the following formulas.

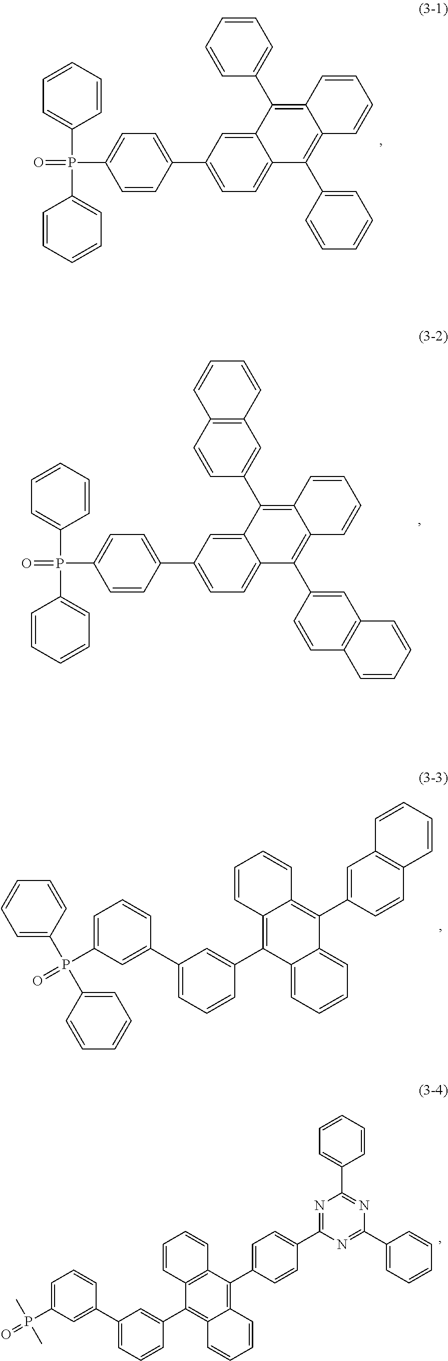 [SMILES: O=P(c1ccccc1)(c1ccccc1)c1ccc(-c2ccc3c(-c4ccccc4)c4ccccc4c(-c4ccccc4)c3c2)cc1 ; O=P(c1ccccc1)(c1ccccc1)c1ccc(-c2ccc3c(-c4ccc5ccccc5c4)c4ccccc4c(-c4ccc5ccccc5c4)c3c2)cc1 ; O=P(c1ccccc1)(c1ccccc1)c1cccc(-c2cccc(-c3c4ccccc4c(-c4ccc5ccccc5c4)c4ccccc34)c2)c1 ; CP(C)(=O)c1cccc(-c2cccc(-c3c4ccccc4c(-c4ccc(-c5nc(-c6ccccc6)nc(-c6ccccc6)n5)cc4)c4ccccc34)c2)c1]

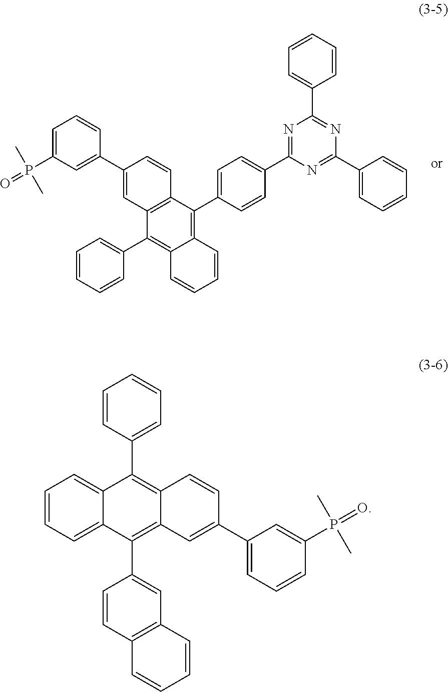 [SMILES: CP(C)(=O)c1cccc(-c2ccc3c(-c4ccc(-c5nc(-c6ccccc6)nc(-c6ccccc6)n5)cc4)c4ccccc4c(-c4ccccc4)c3c2)c1 ; CP(C)(=O)c1cccc(-c2ccc3c(-c4ccccc4)c4ccccc4c(-c4ccc5ccccc5c4)c3c2)c1]

It will be noted that two adjacent ones of R₄, R₅, R₆, R₇, R₈, R₉, R₁₀ and R₁₁ selected from the structural formula of the fourth host material shown in the general formula (III) may bond to form a ring. In a case where h is greater than or equal to 2 (h≥2), adjacent A₃ may bond to form a ring. In a case where k is greater than or equal to 2 (k≥2), adjacent A₄ may bond to form a ring. As for that the two adjacent ones of R₄, R₅, R₆, R₇, R₈, R₉, R₁₀ and R₁₁ bond to form a ring, the adjacent A₃ bond to form a ring and the adjacent A₄ bond to form a ring, reference may be made to the description of B1 and B2 bonding to form a ring shown in the structure in the general formula (II) and in the structure in the general formula (I), and details are not repeated here.

The two adjacent ones of R₄, R₅, R₆, R₇, R₈, R₉, R₁₀ and R₁₁ bonding to form a ring, the adjacent A₃ bonding to form a ring and the adjacent A₄ bonding to form a ring may improve the structural rigidity of the fourth host material, that is, improve the stability of the fourth host material. For example, in a case where the fourth host material is used to form a film layer such as the electron generation layer 31 (as shown in FIG. 1) by evaporation, the structural stability of the material may be ensured, thereby effectively ensuring good performance of the formed film layer.

By testing dipole moments and glass transition temperatures Tg of materials of anthracene derivatives containing phosphorus-oxygen bonds and an anthracene derivative without a phosphorus-oxygen bond (expressed as comparative N-CGL), the relative properties of the two types of materials may be compared, where the structural formula of comparative N-CGL is shown in the following formula.

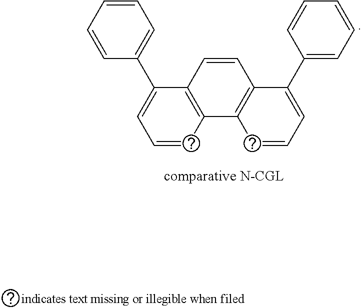 [SMILES: c1ccc(-c2cccc3c2ccc2c(-c4ccccc4)cccc23)cc1]

The dipole moments and the glass transition temperatures Tg of the fourth host materials as shown in the above structural formula (3-4) and structural formula (3-6), and the comparative N-CGL are shown in Table 1 below.

TABLE 1

| | Dipole moment | Tg |
|---|---|---|
| Material | (D) | (° C.) |
| (3-4) | 5.08 | 146 |
| (3-6) | 4.91 | 140 |
| Comparative | 3.96 | 120 |
| N-CGL | | |

It will be noted that a product of a distance between centers of positive and negative charges and the amount of electricity carried by the charge center is called the dipole moment. The larger the dipole moment is, the better the electron injection function of the material is.

The magnitude of the glass transition temperature Tg determines the thermal stability of the material in the evaporation process. The higher the Tg, the better the thermal stability of the material. For example, the glass transition temperature Tg is detected by a differential scanning calorimeter (DSC) in a test environment of the test atmosphere being nitrogen, the heating rate being 10° C./min, and the temperature being in a range of 50° C. to 300° C., inclusive.

Therefore, it can be seen from Table 1 that the fourth host materials shown in the above structural formula (3-4) and structural formula (3-6) are anthracene derivatives containing phosphorus-oxygen bonds, and dipole moments thereof are greater than the dipole moment of the comparative N-CGL, which shows that such materials like the anthracene derivatives containing phosphorus-oxygen bonds have a good electron injection function. Moreover, the glass transition temperature Tg of the anthracene derivatives containing phosphorus-oxygen bonds as shown in the above structural formula (3-4) and structural formula (3-6) are significantly higher than the glass transition temperature Tg of the comparative N-CGL. Therefore, an anthracene derivative containing a phosphorus-oxygen bond, as a material for forming the electron generation layer 31, has good thermal stability.

In some examples, as shown in FIGS. 1 to 3, the dipole moment of the electron generation layer 31 is greater than 4 D. Such a provision may ensure that the electron generation layer 31 has good electron injection properties.

In some embodiments, the electron generation layer 31 further includes a fourth doping material, and the fourth doping material includes any of alkali metals and oxides thereof, alkaline-earth metals and oxides thereof, and transition metals and oxides thereof.

For example, the alkali metals include all metal elements in Group IA of the periodic table of elements, including lithium (Li), sodium (Na), potassium (K), rubidium (Rb), cesium (Cs) and francium (Fr). The oxide of alkali metal is, for example, lithium oxide, sodium oxide, or cesium oxide.

For example, the alkaline-earth metals refer to elements in Group IIA of the periodic table of elements, including beryllium (Be), magnesium (Mg), calcium (Ca), strontium (Sr), barium (Ba) and radium (Ra). The oxide of alkaline-earth metal is, for example, magnesium oxide or barium oxide.

The material of the electron generation layer 31 includes an anthracene derivative containing a phosphorus-oxygen bond as a host material, and a dopant of any of alkali metals and oxides thereof, alkaline-earth metals and oxides thereof, and transition metals and oxides thereof is added to the material of the electron generation layer 31. Thus, the charge injection capability of the electron generation layer 31 may further be improved, and the luminous efficiency of the light-emitting device 10 may be improved.

In some embodiments, as shown in FIGS. 1 to 3, the light-emitting layer 14 includes a first sub-pixel film layer 14a, a second sub-pixel film layer 14b and a third sub-pixel film layer 14c. The first sub-pixel film layer 14a, the second sub-pixel film layer 14b and the third sub-pixel film layer 14c are arranged in a first direction X, and the first direction X is perpendicular to a direction G of light exiting from the light-emitting device 10.

The first sub-pixel film layer 14a is configured to emit one of red light, blue light and green light, the second sub-pixel film layer 14b is configured to emit another one of red light, blue light and green light, and the third sub-pixel film layer 14c is configured to emit the last one of red light, blue light and green light.

For example, the first sub-pixel film layer 14a is configured to emit red light, the second sub-pixel film layer 14b is configured to emit green light, and the third sub-pixel film layer 14c is configured to emit blue light.

In some embodiments, as shown in FIG. 1, the first type of functional layer 21 includes a hole transport layer 12 and a plurality of electron blocking layers 13 disposed between the hole transport layer 12 and the light-emitting layer 14. At least two of the hole transport layer 12 and the plurality of electron blocking layers 13 contain the first type of compounds.

In some examples, as shown in FIG. 1, a first electrode 19 of the first light-emitting unit 1 of the light-emitting device 10 is an anode. The first electrode 19, the hole transport layer 12, the electron blocking layer 13 and the light-emitting layer 14 are arranged in sequence in a second direction Y, the second direction Y is perpendicular to the first direction X, and the second direction Y is parallel to the direction G of the light exiting from the light-emitting device 10. Film layers provided between the anode and the light-emitting layer 14 are used to transport holes to the light-emitting layer 14. At least two of the hole transport layer 12 and the plurality of electron blocking layers 13 are each provided to contain the first type of compound with the hole transport function centered on the sp3 hybridized carbon atom (C), so as to achieve the energy level matching between the hole transport layer 12, the electron blocking layer 13 and the light-emitting layer 14, thereby reducing the hole transport barrier and improving the hole transport efficiency.

In some embodiments, as shown in FIG. 1, the second type of functional layer 41 includes one electron transport functional layer 41a. The electron transport functional layer 41a is a hole blocking layer 15, and the hole blocking layer 15 contains the second type of compound. That is, the second type of compound is an electron transport type material centered on the sp3 hybridized carbon atom (C).

In some embodiments, as shown in FIG. 2, the second type of functional layer 41 includes a plurality of electron transport functional layers 41a. The plurality of electron transport functional layers 41a include a hole blocking layer 15 and an electron transport layer 16. The hole blocking layer 15 and the electron transport layer 16 both contain the second type of compounds.

For example, as shown in FIG. 2, the first electrode 19 of the first light-emitting unit 1 of the light-emitting device 10 is an anode. The first electrode 19, the hole transport layer 12, the electron blocking layer 13, the light-emitting layer 14, the hole blocking layer 15 and the electron transport layer 16 are arranged in sequence in a second direction Y. At least two of the hole transport layer 12 and the plurality of electron blocking layers 13 are each provided to contain the first type of compound centered on the sp3 hybridized carbon atom (C), and the hole blocking layer 15 and the electron transport layer 16 each contain the second type of compound centered on the sp3 hybridized carbon atom (C). In this way, adjacent functional layers in the light-emitting device 10 may achieve optimal matching, so that transport of electrons in the second type of functional layer 41 and transport of holes in the first type of functional layer 21 are caused to be easy, so as to ensure that electrons and holes recombine in the light-emitting layer 14 to form excitons and then emit light, thereby improving the luminous efficiency of the light-emitting device 10.

In some embodiments, as shown in FIGS. 1 and 2, the plurality of electron blocking layers 13 includes a first electron blocking layer 13a, a second electron blocking layer 13b, and a third electron blocking layer 13c. The first electron blocking layer 13a, the second electron blocking layer 13b and the third electron blocking layer 13c are arranged in the first direction X.

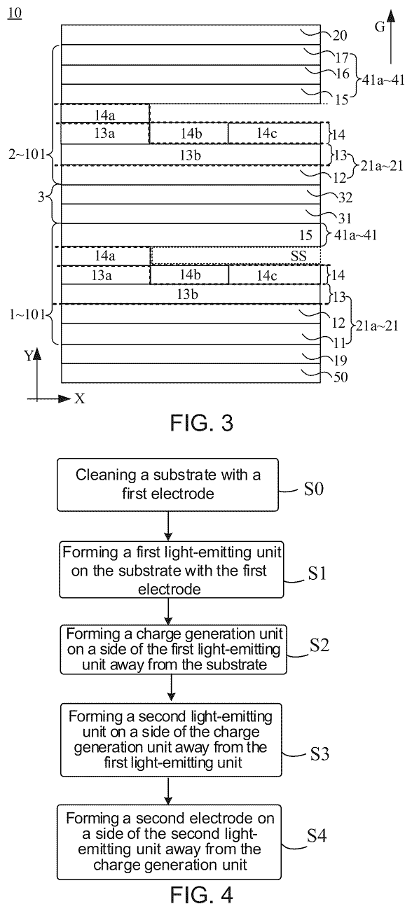

Alternatively, as shown in FIG. 3, the plurality of electron blocking layers 13 include a first electron blocking layer 13a and a second electron blocking layer 13b. The second electron blocking layer 13b is disposed between the light-emitting layer 14 and the hole transport layer 12. The first electron blocking layer 13a is disposed between the second electron blocking layer 13b and the first sub-pixel film layer 14a.

In some examples, as shown in FIGS. 1 and 2, the electron blocking layers 13 of the first light-emitting unit 1 and the second light-emitting unit 2 of the light-emitting device 10 each include a first electron blocking layer 13a, a second electron blocking layer 13b and a third electron blocking layer 13c arranged in the first direction X. The light-emitting layer 14 includes a first sub-pixel film layer 14a, a second sub-pixel film layer 14b and a third sub-pixel film layer 14c arranged in the first direction X. The first electron blocking layer 13a is disposed correspondingly to the first sub-pixel film layer 14a, the second electron blocking layer 13b is disposed correspondingly to the second sub-pixel film layer 14b, and the third electron blocking layer 13c is disposed correspondingly to the third sub-pixel film layer 14c.

It will be noted that the description of “the first electron blocking layer 13a being disposed correspondingly to the first sub-pixel film layer 14a” means that an orthographic projection of the first sub-pixel film layer 14a on the hole transport layer 12 coincides with an orthographic projection of the first electron blocking layer 13a on the hole transport layer 12. Regarding the understanding of the second electron blocking layer 13b being disposed correspondingly to the second sub-pixel film layer 14b and the third electron blocking layer 13c being disposed correspondingly to the third sub-pixel film layer 14c, reference may be made to the description of the corresponding relationship between the first sub-pixel film layer 14a and the first electron blocking layer 13a, and details are not repeated here.

In some examples, as shown in FIG. 3, the electron blocking layers 13 of the first light-emitting unit 1 and the second light-emitting unit 2 of the light-emitting device 10 each include a first electron blocking layer 13a and a second electron blocking layer 13b. The second electron blocking layer 13b and the first electron blocking layer 13a are provided in sequence between the hole transport layer 12 and the first sub-pixel film layer 14a, and the second electron blocking layer 13b and the first electron blocking layer 13a are disposed in a direction from the hole transport layer 12 to the first sub-pixel film layer 14a. The second electron blocking layer 13b is provided as a whole layer, and the first electron blocking layer 13a is only provided between the second electron blocking layer 13b and the first sub-pixel film layer 14a. With such a provision, when the second sub-pixel film layer 14b is lit up, the first sub-pixel film layer 14a may be prevented from being lit up.

For example, the first sub-pixel film layer 14a is configured to emit red light. The turn-on voltages of the sub-pixel film layers of light of different colors have the following relationship: blue>green>red. Therefore, the first electron blocking layer 13a and the second electron blocking layer 13b are disposed between the first sub-pixel film layer 14a and the hole transport layer 12, and the second electron blocking layer 13b is disposed between both the second sub-pixel film layer 14b and the third sub-pixel film layer 14c and the hole transport layer 12, so that the first sub-pixel film layer 14a may be prevented from being mistakenly lit up, thereby avoiding cross-color interference.

In some embodiments, as shown in FIGS. 1 and 2, the first sub-pixel film layer 14a is configured to emit red light. The first electron blocking layer 13a is disposed between the first sub-pixel film layer 14a and the hole transport layer 12, and the first electron blocking layer 13a and the hole transport layer 12 each contain the first type of compound.

In some examples, as shown in FIG. 1, the first type of functional layers 21 of the first light-emitting unit 1 and the second light-emitting unit 2 of the light-emitting device 10 each includes a first electron blocking layer 13a, a second electron blocking layer 13b, a third electron blocking layer 13c and the hole transport layer 12. At least two hole transport functional layers 21a in the first type of functional layer 21 contain the first type of compound centered on the sp3 hybridized carbon atom (C), and the at least two hole transport functional layers 21a are the first electron blocking layer 13a and the hole transport layer 12.

In some embodiments, as shown in FIGS. 1 and 2, the first sub-pixel film layer 14a is configured to emit red light, and the first electron blocking layer 13a is disposed between the first sub-pixel film layer 14a and the hole transport layer 12. The second sub-pixel film layer 14b is configured to emit green light, and the second electron blocking layer 13b is disposed between the second sub-pixel film layer 14b and the hole transport layer 12. The first electron blocking layer 13a and the second electron blocking layer 13b each contain the first type of compound.

In some examples, as shown in FIG. 1, the first type of functional layers 21 of the first light-emitting unit 1 and the second light-emitting unit 2 of the light-emitting device 10 each includes a first electron blocking layer 13a, a second electron blocking layer 13b, a third electron blocking layer 13c and the hole transport layer 12. At least two hole transport functional layers 21a in the first type of functional layer 21 contain the first type of compound centered on the sp3 hybridized carbon atom (C), and the at least two hole transport functional layers 21a are the first electron blocking layer 13a and the second electron blocking layer 13b.

In some embodiments, as shown in FIGS. 1 and 2, the plurality of electron blocking layers 13 include a first electron blocking layer 13a, a second electron blocking layer 13b and a third electron blocking layer 13c. The first sub-pixel film layer 14a is configured to emit red light, and the first electron blocking layer 13a is disposed between the first sub-pixel film layer 14a and the hole transport layer 12. The second sub-pixel film layer 14b is configured to emit green light, and the second electron blocking layer 13b is disposed between the second sub-pixel film layer 14b and the hole transport layer 12. The third sub-pixel film layer 14c is configured to emit blue light, and the third electron blocking layer 13c is disposed between the third sub-pixel film layer 14c and the hole transport layer 12. A specific surface area of the first electron blocking layer 13a is smaller than a specific surface area of the second electron blocking layer 13b, and the specific surface area of the first electron blocking layer 13a is smaller than a specific surface area of the third electron blocking layer 13c.

In some examples, as shown in FIG. 1, the electron blocking layers 13 of the first and second light-emitting units 1 and 2 of the light-emitting device 10 each include: a first electron blocking layer 13a, a second electron blocking layer 13b and a third Electron blocking layer 13c. The specific surface area of the first electron blocking layer 13a is smaller than the specific surface area of the second electron blocking layer 13b, and the specific surface area of the first electron blocking layer 13a is smaller than the specific surface area of the third electron blocking layer 13c.

The turn-on voltage of the third sub-pixel film layer 14c is greater than the turn-on voltage of the second sub-pixel film layer 14b, and the turn-on voltage of the second sub-pixel film layer 14b is greater than the turn-on voltage of the first sub-pixel film layer 14a. That is, the turn-on voltages have the following relationship: blue>green>red.

The specific surface area may reflect the lateral resistance of the material to a certain extent. The smaller the specific surface area, the greater the lateral resistance. The first sub-pixel film layer 14a emits red light and has a low turn-on voltage. By setting the specific surface area of the first sub-pixel film layer 14a smallest, the resistance of the first sub-pixel film layer 14a increases, and thus the color crosstalk may be suppressed.

In some embodiments, as shown in FIGS. 1 to 3, the at least two light-emitting units 101 include a first light-emitting unit 1 and a second light-emitting unit 2. The first light-emitting unit 1, the charge generation unit 3 and the second light-emitting unit 2 are stacked in sequence in the second direction Y, and the second direction Y is perpendicular to the first direction X.

The first type of functional layer 21, the light-emitting layer 14 and the second type of functional layer 41 of the first light-emitting unit 1 are stacked in the second direction Y The first type of functional layer of the first light-emitting unit 1 further includes a hole injection layer 11, and the hole injection layer 11 is disposed on a side of the hole transport layer 12 away from the light-emitting layer 14.

The first type of functional layer 21, the light-emitting layer 14 and the second type of functional layer 41 of the second light-emitting unit 2 are stacked in the second direction Y The second type of functional layer 41 of the second light-emitting unit 2 includes a hole blocking layer 15 and an electron transport layer 16, and the second type of functional layer 41 of the second light-emitting unit 2 further include an electron injection layer 17. The electron injection layer 17 is disposed on a side of the electron transport layer 16 away from the light-emitting layer 14.

In some embodiments, as shown in FIGS. 1 to 3, the light-emitting device 10 further includes a first electrode 19 and a second electrode 20. The at least two light-emitting units 101 and the charge generation unit 3 are disposed between the first electrode 19 and the second electrode 20.

Based on the above introduction of the structure of the light-emitting device 10, three embodiments of the structure of the light-emitting device 10 are provided.

As shown in FIGS. 1 to 3, the light-emitting device 10 includes a first electrode 19, a first light-emitting unit 1, a charge generation unit 3, a second light-emitting unit 2 and a second electrode 20 that are sequentially arranged in the second direction Y.

### Embodiment 1

The light-emitting device 10 has a structure shown in FIG. 1.

The first light-emitting unit 1 of the light-emitting device 10 includes a hole injection layer 11, a hole transport layer 12, an electron blocking layer 13, a light-emitting layer 14 and a hole blocking layer 15.

The second light-emitting unit 2 of the light-emitting device 10 includes a hole transport layer 12, an electron blocking layer 13, a light-emitting layer 14, a hole blocking layer 15, an electron transport layer 16 and an electron injection layer 17.

The electron generation layer 31 of the charge generation unit 3 is used to inject electrons into the first light-emitting unit 1, and the hole generation layer 32 of the charge generation unit 3 is used to inject holes into the second light-emitting unit 2.

The light-emitting device 10 includes a first electrode 19, the hole injection layer 11 (5 nm to 30 nm), the hole transport layer 12 (15 nm to 25 nm), the electron blocking layer 13, the light-emitting layer 14, the hole blocking layer 15 (5 nm to 15 nm), the electron generation layer 31 (15 nm to 25 nm), the hole generation layer 32 (5 nm to 15 nm), the hole transport layer 12 (15 nm to 25 nm), the electron blocking layer 13, the light-emitting layer 14, the hole blocking layer 15 (5 nm to 15 nm), the electron transport layer 16 (20 nm to 100 nm), the electron injection layer 17 (1 nm to 10 nm) and a second electrode 20 (10 nm to 20 nm) that are arranged in sequence in the second direction Y.

The electron blocking layers 13 of the first light-emitting unit 1 and the second light-emitting unit 2 of the light-emitting device 10 each includes a first electron blocking layer 13a (5 nm to 45 nm), a second electron blocking layer 13b (10 nm to 25 nm) and a third electron blocking layer 13c (5 nm to 15 nm). The light-emitting layers 14 of the first light-emitting unit 1 and the second light-emitting unit 2 of the light-emitting device 10 each include a first sub-pixel film layer 14a (30 nm to 50 nm), a second sub-pixel film layer 14b (30 nm to 50 nm) and a third sub-pixel film layer 14c (10 nm to 20 nm).

It will be noted that values in a bracket after the film layer refers to a thickness range of the film layer. For example, the first electron blocking layer 13a (5 nm to 45 nm) means that the thickness range of the first electron blocking layer 13a is from 5 nm to 45 nm. As shown in FIG. 1, the thickness refers to a dimension of a film layer in the second direction Y For example, the dimension of the first electron blocking layer 13a in the second direction Y may be represented as a dimension d1. For the thickness ranges of other film layers, reference may be made to the introduction of the thickness range of the first electron blocking layer 13a, and details are not repeated here.

For example, as shown in FIG. 1, the thickness of each film layer of the light-emitting device 10 is as following: the hole injection layer 11 (10 nm)/the hole transport layer 12 (19 nm)/the first electron blocking layer 13a (25 nm)/the second electron blocking layer 13b (15 nm)/the third electron blocking layer 13c (5 nm)/the first sub-pixel film layer 14a (3 wt %, 42 nm)/the second sub-pixel film layer 14b (10 wt %, 40 nm)/the third sub-pixel film layer 14c (3 wt %, 15 nm)/the hole blocking layer 15 (5 nm)/the electron generation layer 31 (1 wt % Yb, 18 nm)/the hole generation layer 32 (5 wt %, 9 nm)/the hole transport layer 12 (19 nm)/the first electron blocking layer 13a (25 nm)/the second electron blocking layer 13b (15 nm)/the third electron blocking layer 13c (5 nm)/the first sub-pixel film layer 14a (3 wt %, 42 nm)/the second sub-pixel film layer 14b (10 wt %, 40 nm)/the third sub-pixel film layer 14c (3 wt %, 15 nm)/the hole blocking layer 15 (5 nm)/the electron transport layer 16 (50 wt % LiQ, 35 nm)/the electron injection layer 17 (1 nm)/the second electrode 20 (15 nm).

In the first sub-pixel film layer 14a (3 wt %, 42 nm), 3 wt % refers to that a mass proportion of a first doping material in the first sub-pixel film layer 14a is 3%, and 42 nm refers to that a thickness of the first sub-pixel film layer 14a is 42 nm. Similarly, in the second sub-pixel film layer 14b (10 wt %, 40 nm) 10 wt % refers to that a mass proportion of a second doping material in the second sub-pixel film layer 14b is 10%, and 40 nm refers to that a thickness of the second sub-pixel film layer 14b is 40 nm. In the third sub-pixel film layer 14c (3 wt %, 15 nm), 3 wt % refers to that a mass proportion of a third doping material in the third sub-pixel film layer 14c is 3%, and 15 nm refers to that a thickness of the third sub-pixel film layer 14c is 15 nm.

In the electron generation layer 31 (1 wt % Yb, 18 nm), 1 wt % Yb refers to that the fourth doping material in the electron generation layer 31 is ytterbium (Yb), and a mass proportion of the fourth doping material in the electron generation layer 31 is 3%. In the electron transport layer 16 (50 wt % LiQ, 35 nm), 50 wt % LiQ refers to that a mass proportion of 8-hydroxyquinolinolato-lithium (LiQ) in the electron transport layer 16 is 50%, that is, a mass ratio of the second type of compound to the 8-hydroxyquinolinolato-lithium (LiQ) in the electron transport layer 16 is 1:1. The structural formula of the 8-hydroxvquinolinolato-lithium (LiQ) is as follows.

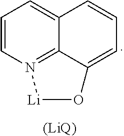

For example, eight film layers of the hole transport layer 12 of the first light-emitting unit 1, the first electron blocking layer 13a of the first light-emitting unit 1, the hole blocking layer 15 of the first light-emitting unit 1, the hole generation layer 32, the hole transport layer 12 of the second light-emitting unit 2, the first electron blocking layer 13a of the second light-emitting unit 2, the hole blocking layer 15 of the second light-emitting unit 2, and the electron transport layer 16 of the second light-emitting unit 2 are each provided to contain a compound centered on an sp3 hybridized carbon atom (C). Then, the first light-emitting unit 1, the charge generation unit 3, and the second light-emitting unit 2 of the light-emitting device 10 all contain the compounds centered on the sp3 hybridized carbon atom (C).

That is, the hole transport layer 12 of the first light-emitting unit 1, the first electron blocking layer 13a of the first light-emitting unit 1, the hole generation layer 32, the hole transport layer 12 of the second light-emitting unit 2, the first electron blocking layer 13a of the second light-emitting unit 2 are each provided as a film layer containing the first type of compound; and the hole blocking layer 15 of the first light-emitting unit 1, the hole blocking layer 15 of the second light-emitting unit 2, and the electron transport layer 16 of the second light-emitting unit 2 are each provided as a film layer containing the second type of compound.

Such a design makes the hole generation layer 32 and other functional layers (e.g., the first type of functional layer 21 and the second type of functional layer 41) in the stacked light-emitting device 10 contain similar chemical structures, which is conducive to control of charges and balance of charge transport. With such the design, the charges may be smoothly injected into the light-emitting layers 14 located on both sides of the charge generation unit 3; moreover, it is conducive to control regions of exciton recombination in the upper and lower light-emitting units 101. By reasonably matching materials in the charge generation unit 3 and other functional layers, centers of exciton recombination in the light-emitting layers 14 located on both sides of the charge generation unit 3 may be close to middles of the light-emitting layers 14, thereby helping to improve the utilization rate of excitons.

For example, seven film layers of the first electron blocking layer 13a of the first light-emitting unit 1, the second electron blocking layer 13b of the first light-emitting unit 1, the hole blocking layer 15 of the first light-emitting unit 1, the first electron blocking layer 13a of the second light-emitting unit 2, the second electron blocking layer 13b of the second light-emitting unit 2, the hole blocking layer 15 of the second light-emitting unit 2 and the electron transport layer 16 of the second light-emitting unit 2 are each provided to contain a compound centered on an sp3 hybridized carbon atom (C). Then, the first light-emitting unit 1 and the second light-emitting unit 2 of the light-emitting device 10 contain the compounds centered on the sp3 hybridized carbon atom (C).

That is, the first electron blocking layer 13a of the first light-emitting unit 1, the second electron blocking layer 13b of the first light-emitting unit 1, the first electron blocking layer 13a of the second light-emitting unit 2 and the second electron blocking layer 13b of the second light-emitting unit 2 are each provided as a film layer containing the first type of compound; and the hole blocking layer 15 of the first light-emitting unit 1, the hole blocking layer 15 of the second light-emitting unit 2 and the electron transport layer 16 of the second light-emitting unit 2 are each provided as a film layer containing the second type of compound.

Such a design is also conducive to the balance of charge transport, and may make the centers of exciton recombination in the light-emitting layers 14 located on both sides of the charge generation unit 3 may be close to middles of the light-emitting layers 14, thereby helping to improve the utilization rate of excitons.

It will be noted that the structures of the first type of compound and the second type of compound may refer to the above contents, and details are not repeated here.

In the present embodiment, the hole transport layers 12, the first electron blocking layers 13a, the second electron blocking layers 13b and the third electron blocking layers 13c of the first light-emitting unit 1 and the second light-emitting unit 2 are set to be the same in thickness, which is only an example, but not limitations on the embodiment. It will be noted that the hole transport layers 12, the first electron blocking layers 13a, the second electron blocking layers 13b and the third electron blocking layers 13c of the first light-emitting unit 1 and the second light-emitting unit 2 may be different in thickness and material.

### Embodiment 2

The light-emitting device 10 has a structure shown in FIG. 2.

The first light-emitting unit 1 of the light-emitting device 10 includes a hole injection layer 11, a hole transport layer 12, an electron blocking layer 13, a light-emitting layer 14, a hole blocking layer 15 and an electron transport layer 16.

The second light-emitting unit 2 of the light-emitting device 10 includes a hole transport layer 12, an electron blocking layer 13, a light-emitting layer 14, a hole blocking layer 15, an electron transport layer 16 and an electron injection layer 17.

The light-emitting device 10 includes a first electrode 19, the hole injection layer 11, the hole transport layer 12, the electron blocking layer 13, the light-emitting layer 14, the hole blocking layer 15, the electron transport layer 16, the electron generation layer 31, the hole generation layer 32, the hole transport layer 12, the electron blocking layer 13, the light-emitting layer 14, the hole blocking layer 15, the electron transport layer 16, the electron injection layer 17 and a second electrode 20 that are arranged in sequence in the second direction Y.

The electron blocking layers 13 of the first light-emitting unit 1 and the second light-emitting unit 2 of the light-emitting device 10 each includes a first electron blocking layer 13a, a second electron blocking layer 13b and a third electron blocking layer 13c. The light-emitting layers 14 of the first light-emitting unit 1 and the second light-emitting unit 2 of the light-emitting device 10 each includes a first sub-pixel film layer 14a, a second sub-pixel film layer 14b and a third sub-pixel film layer 14c.

For example, nine film layers of the hole transport layer 12 of the first light-emitting unit 1, the first electron blocking layer 13a of the first light-emitting unit 1, the hole blocking layer 15 of the first light-emitting unit 1, the electron transport layer 16 of the first light-emitting unit 1, the hole generation layer 32, the hole transport layer 12 of the second light-emitting unit 2, the first electron blocking layer 13a of the second light-emitting unit 2, the hole blocking layer 15 of the second light-emitting unit 2, and the electron transport layer 16 of the second light-emitting unit 2 are each provided to contain a compound centered on an sp3 hybridized carbon atom (C).

That is, the hole transport layer 12 of the first light-emitting unit 1, the first electron blocking layer 13a of the first light-emitting unit 1, the hole generation layer 32, the hole transport layer 12 of the second light-emitting unit 2 and the first electron blocking layer 13a of the second light-emitting unit 2 are each provided as a film layer containing the first type of compound; and the hole blocking layer 15 of the first light-emitting unit 1, the electron transport layer 16 of the first light-emitting unit 1, the hole blocking layer 15 of the second light-emitting unit 2 and the electron transport layer 16 of the second light-emitting unit 2 are each provided as a film layer containing the second type of compound.

For example, eight film layers of the first electron blocking layer 13a of the first light-emitting unit 1, the second electron blocking layer 13b of the first light-emitting unit 1, the hole blocking layer 15 of the first light-emitting unit 1, the electron transport layer 16 of the first light-emitting unit 1, the first electron blocking layer 13a of the second light-emitting unit 2, the second electron blocking layer 13b of the second light-emitting unit 2, the hole blocking layer 15 of the second light-emitting unit 2 and the electron transport layer 16 of the second light-emitting unit 2 are each provided to contain a compound centered on an sp3 hybridized carbon atom (C).

That is, the first electron blocking layer 13a of the first light-emitting unit 1, the second electron blocking layer 13b of the first light-emitting unit 1, the first electron blocking layer 13a of the second light-emitting unit 2 and the second electron blocking layer 13b of the second light-emitting unit 2 are each provided as a film layer containing the first type of compound; and the hole blocking layer 15 of the first light-emitting unit 1, the electron transport layer 16 of the first light-emitting unit 1, the hole blocking layer 15 of the second light-emitting unit 2 and the electron transport layer 16 of the second light-emitting unit 2 are each provided as a film layer containing the second type of compound.

Such a design is conducive to the balance of charge transport, and may make the centers of exciton recombination in the light-emitting layers 14 located on both sides of the charge generation unit 3 may be close to middles of the light-emitting layers 14, thereby helping to improve the utilization rate of excitons.

In the present embodiment, the electron transport layer 16 is added to the first light-emitting unit 1. The electron transport layer 16 may further increase the efficiency of electron transport and further improve the luminous performance of the light-emitting device 10.

It will be noted that as for the thickness of each film layer in the light-emitting device 10 provided in the present embodiment, reference may be made to Embodiment 1, and details are not repeated here.

### Embodiment 3

The light-emitting device 10 has a structure shown in FIG. 3.

The first light-emitting unit 1 of the light-emitting device 10 includes a hole injection layer 11, a hole transport layer 12, an electron blocking layer 13, a light-emitting layer 14 and a hole blocking layer 15.

The second light-emitting unit 2 of the light-emitting device 10 includes a hole transport layer 12, an electron blocking layer 13, a light-emitting layer 14, a hole blocking layer 15, an electron transport layer 16 and an electron injection layer 17.

The light-emitting device 10 includes a first electrode 19, the hole injection layer 11, the hole transport layer 12, the electron blocking layer 13, the light-emitting layer 14, the hole blocking layer 15, the electron generation layer 31, the hole generation layer 32, the hole transport layer 12, the electron blocking layer 13, the light-emitting layer 14, the hole blocking layer 15, the electron transport layer 16, the electron injection layer 17 and a second electrode 20 that are arranged in sequence in the second direction Y.

The light-emitting layers 14 of the first light-emitting unit 1 and the second light-emitting unit 2 of the light-emitting device 10 each includes a first sub-pixel film layer 14a, a second sub-pixel film layer 14b and a third sub-pixel film layer 14c.

The electron blocking layers 13 of the first light-emitting unit 1 and the second light-emitting unit 2 of the light-emitting device 10 each includes a first electron blocking layer 13a and a second electron blocking layer 13b. Moreover, the second electron blocking layer 13b is provided as a whole layer, and the first electron blocking layer 13a is only provided between the second electron blocking layer 13b and the first sub-pixel film layer 14a.

For example, eight film layers of the hole transport layer 12 of the first light-emitting unit 1, the first electron blocking layer 13a of the first light-emitting unit 1, the hole blocking layer 15 of the first light-emitting unit 1, the hole generation layer 32, the hole transport layer 12 of the second light-emitting unit 2, the first electron blocking layer 13a of the second light-emitting unit 2, the hole blocking layer 15 of the second light-emitting unit 2, and the electron transport layer 16 of the second light-emitting unit 2 are each provided to contain a compound centered on an sp3 hybridized carbon atom (C).

For example, seven film layers of the first electron blocking layer 13a of the first light-emitting unit 1, the second electron blocking layer 13b of the first light-emitting unit 1, the hole blocking layer 15 of the first light-emitting unit 1, the first electron blocking layer 13a of the second light-emitting unit 2, the second electron blocking layer 13b of the second light-emitting unit 2, the hole blocking layer 15 of the second light-emitting unit 2 and the electron transport layer 16 of the second light-emitting unit 2 are each provided to contain a compound centered on an sp3 hybridized carbon atom (C).

For the material of each film layer in the present embodiment, reference may be made to selection of a compound centered on an sp3 hybridized carbon atom (C) for each film layer in Embodiment 1, and details are not repeated here.

It will be noted that for the thickness of each film layer of the light-emitting device 10 provided in the present embodiment, reference may be made to Embodiment 1, and details are not repeated here.

It will be noted that FIG. 3 does not actually show the stacked structure of all film layers of the light-emitting device 10, but is only used to illustrate the order in which all the film layers are stacked. Since the second electron blocking layer 13b is provided as a whole layer, the first electron blocking layer 13a is only provided between the second electron blocking layer 13b and the first sub-pixel film layer 14a. Therefore, in FIG. 3, a gap region SS is shown between the light-emitting layer 14 and the adjacent hole blocking layer 15, but actually there is no such the gap region SS in the structure of the light-emitting device 10. During manufacturing the light-emitting device 10, a film layer formed later may cover the former film layer. For example, the hole blocking layer 15 formed later may directly cover the light-emitting layer 14.

Embodiment 3 provided by the present disclosure may be not only beneficial to the balance of charge transport, so as to make the centers of exciton recombination in the light-emitting layers 14 located on both sides of the charge generation unit 3 may be close to middles of the light-emitting layers 14, thereby improving the utilization rate of excitons, but also further prevent the first sub-pixel film layer 14a from being mistakenly lit up, thereby avoiding cross-color interference.

Therefore, the light-emitting devices 10 provided in the above Embodiment 1 to Embodiment 3 may ensure smooth generation, injection, and transport of charges, and achieve balance in the light-emitting layer 14, thereby improving the efficiency of the light-emitting device 10 significantly.

It will be noted that the light-emitting devices 10 provided in the above Embodiment 1 to Embodiment 3 are examples of arrangement of each film layer of the light-emitting device 10, and are not limitations on the structure of the light-emitting device 10.

In some embodiments, as shown in FIGS. 1 to 3, the sub-pixel film layer of the first light-emitting unit 1 and the sub-pixel film layer of the second light-emitting unit 2 emit light of the same color. A difference between the wavelength of the light emitted by the sub-pixel film layer of the first light-emitting unit 1 and the wavelength of the light emitted by the sub-pixel film layer of the second light-emitting unit 2 is less than or equal to 20 nm. The sub-pixel film layer includes any of the first sub-pixel film layer 14a, the second sub-pixel film layer 14b and the third sub-pixel film layer 14c.

For example, the first sub-pixel film layer 14a of the first light-emitting unit 1 and the first sub-pixel film layer 14a of the second light-emitting unit 2 are configured to emit red light, and a difference between the wavelength of the red light emitted by the first sub-pixel film layer 14a of the first light-emitting unit 1 and the wavelength of the red light emitted by the first sub-pixel film layer 14a of the second light-emitting unit 2 is less than or equal to 20 nm.

For example, the second sub-pixel film layer 14b of the first light-emitting unit 1 and the second sub-pixel film layer 14b of the second light-emitting unit 2 are configured to emit green light, and a difference between the wavelength of the green light emitted by the second sub-pixel film layer 14b of the first light-emitting unit 1 and the wavelength of the green light emitted by the second sub-pixel film layer 14b of the second light-emitting unit 2 is less than or equal to 20 nm.

For example, the third sub-pixel film layer 14c of the first light-emitting unit 1 and the third sub-pixel film layer 14c of the second light-emitting unit 2 are configured to emit blue light, and a difference between the wavelength of the blue light emitted by the third sub-pixel film layer 14c of the first light-emitting unit 1 and the wavelength of the blue light emitted by the third sub-pixel film layer 14c of the second light-emitting unit 2 is less than or equal to 20 nm.

For example, the difference between wavelengths of lights emitted by the sub-pixel film layers for emitting light of the same color in the light-emitting layer 14 of the first light-emitting unit 1 and the light-emitting layer 14 of the second light-emitting unit 2 is 5 nm, 10 nm, 15 nm or 20 nm, and is not limited here.

By setting the difference between wavelengths of lights emitted by the sub-pixel film layers for emitting light of the same color in the light-emitting layer 14 of the first light-emitting unit 1 and the light-emitting layer 14 of the second light-emitting unit 2 less than or equal to 20 nm, color separation caused by the microcavity effect may be avoided, thereby avoiding color cast.

In some embodiments, as shown in FIGS. 1 to 3, a ratio of the mobility of the hole blocking layer 15 of the first light-emitting unit 1 to the mobility of the hole blocking layer 15 of the second light-emitting unit 2 is less than or equal to 10 and greater than or equal to 0.1. A ratio of the mobility of the hole transport layer 12 of the first light-emitting unit 1 to the mobility of the hole transport layer 12 of the second light-emitting unit 2 is less than or equal to 10 and greater than or equal to 0.1.

For example, the ratio of the mobility of the hole blocking layer 15 of the first light-emitting unit 1 to the mobility of the hole blocking layer 15 of the second light-emitting unit 2 is 10, 7, 4, 2, 1, 0.5 or 0.1, which is not limited here.

For example, the ratio of the mobility of the hole transport layer 12 of the first light-emitting unit 1 to the mobility of the hole transport layer 12 of the second light-emitting unit 2 is 10, 8, 5, 3, 1, 0.6 or 0.1, which is not limited here.

By setting the ratio of the mobility of the hole blocking layer 15 of the first light-emitting unit 1 to the mobility of the hole blocking layer 15 of the second light-emitting unit 2 less than or equal to 10 and greater than or equal to 0.1, and setting the ratio of the mobility of the hole transport layer 12 of the first light-emitting unit 1 to the mobility of the hole transport layer 12 of the second light-emitting unit 2 less than or equal to 10 and greater than or equal to 0.1, the region of recombination of electrons and holes in the first light-emitting unit 1 is located in the middle of the light-emitting layer 14 of the first light-emitting unit 1, and the region of recombination of electrons and holes in the second light-emitting unit 2 is located in the middle of the light-emitting layer 14 of the second light-emitting unit 2, so that the light-emitting device 10 has a good luminous effect.

In some embodiments, as shown in FIGS. 1 to 3, a difference between the HOMO energy level of the hole generation layer 32 and the HOMO energy level of the hole transport layer 12 of the second light-emitting unit 2 is less than or equal to 0.3 eV. A difference between the LUMO energy level of the electron generation layer 31 and the LUMO energy level of the hole blocking layer 15 of the first light-emitting unit 1 is less than or equal to 0.5 eV.

For example, the difference between the HOMO energy level of the hole generation layer 32 and the HOMO energy level of the hole transport layer 12 of the second light-emitting unit 2 is 0.3 eV, 0.2 eV, 0.1 eV or 0 eV, which is not limited here.

For example, the difference between the LUMO energy level of the electron generation layer 31 and the LUMO energy level of the hole blocking layer 15 of the first light-emitting unit 1 is 0.5 eV, 0.3 eV, 0.1 eV or 0 eV, which is not limited here.

By setting the difference between the HOMO energy level of the hole generation layer 32 and the HOMO energy level of the hole transport layer 12 of the second light-emitting unit 2 less than or equal to 0.3 eV, the energy level transport barrier may be lowered and the hole transport efficiency may be enhanced. By setting the difference between the LUMO energy level of the electron generation layer 31 and the LUMO energy level of the hole blocking layer 15 of the first light-emitting unit 1 less than or equal to 0.5 eV, the energy level transport barrier may also be lowered and the electron transport efficiency may be enhanced.

In some embodiments, as shown in FIGS. 1 to 3, the first sub-pixel film layer 14a includes at least one first host material and a first doping material. The at least one first host material includes two first host materials, and each of the two first host materials is any of exciplexes, isomers and homologues.

The second sub-pixel film layer 14b includes at least two second host materials and a second doping material. The at least two second host materials include two second host materials, and each of the two second host materials is any of exciplexes, isomers and homologues.

The third sub-pixel film layer 14c includes at least one third host material and a third doping material. The at least one third host material includes two third host materials, and each of the two third host materials is any of exciplexes, isomers and homologues. The third host material in the at least one third host material contains an anthracene derivative.

For example, the first sub-pixel film layer 14a is configured to emit red light. The at least one first host material of the first sub-pixel film layer 14a includes two first host materials, and the two first host materials are materials as shown in the following structural formulas (RH-1) and (RH-2).

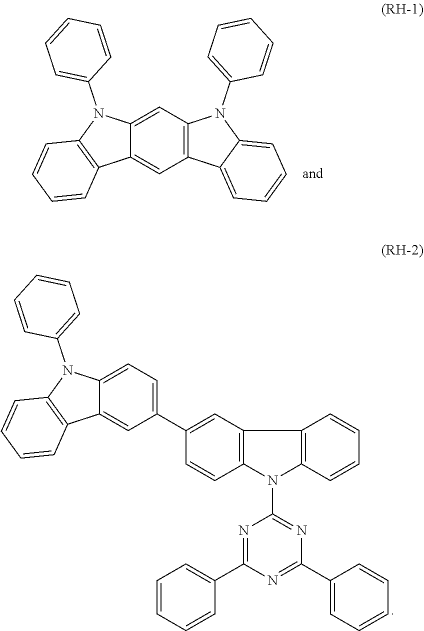 [SMILES: c1ccc(-n2c3ccccc3c3cc4c5ccccc5n(-c5ccccc5)c4cc32)cc1 ; c1ccc(-c2nc(-c3ccccc3)nc(-n3c4ccccc4c4cc(-c5ccc6c(c5)c5ccccc5n6-c5ccccc5)ccc43)n2)cc1]

The two first host materials, as shown in the structural formulas (RH-1) and (RH-2), may form exciplexes.

For example, the first sub-pixel film layer 14a includes a first host material, and the first host material is a material as shown in the following structural formula (RH-3).

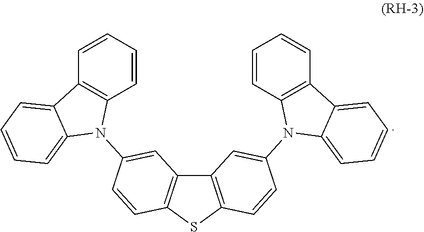 [SMILES: c1ccc2c(c1)c1ccccc1n2-c1ccc2sc3ccc(-n4c5ccccc5c5ccccc54)cc3c2c1]

For example, the second sub-pixel film layer 14b is configured to emit green light. The two second host materials of the second sub-pixel film layer 14b are materials as shown in the following structural formulas (GH-1) and (GH-2).

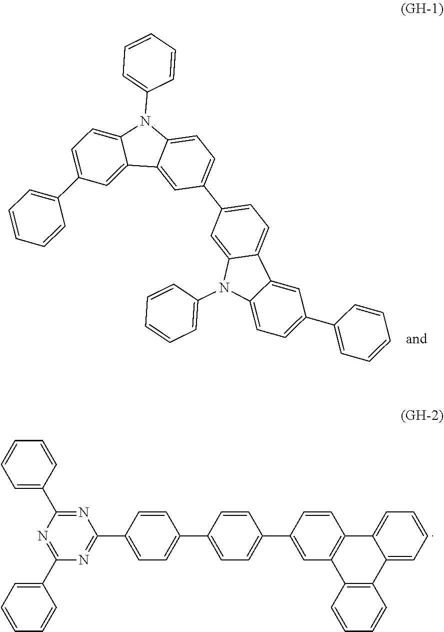 [SMILES: c1ccc(-c2ccc3c(c2)c2cc(-c4ccc5c6cc(-c7ccccc7)ccc6n(-c6ccccc6)c5c4)ccc2n3-c2ccccc2)cc1 ; c1ccc(-c2nc(-c3ccccc3)nc(-c3ccc(-c4ccc(-c5ccc6c7ccccc7c7ccccc7c6c5)cc4)cc3)n2)cc1]

The two second host materials, as shown in the structural formulas (GH-1) and (GH-2), may form exciplexes.

For example, the third sub-pixel film layer 14c is configured to emit blue light. The third sub-pixel film layer 14c includes a third host material, and the third host material is a material as shown in the following structural formula (BH-1). The third host material with the structural formula (BH-1) is generally abbreviated as ADN.

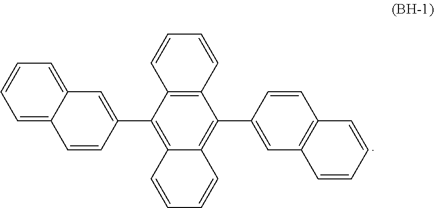 [SMILES: c1ccc2cc(-c3c4ccccc4c(-c4ccc5ccccc5c4)c4ccccc34)ccc2c1]

For example, the third sub-pixel film layer 14c includes a plurality of third host materials, and at least one of the plurality of third host materials is an anthracene derivative. The structure of anthracene is shown in the following formula. It can be understood that the anthracene derivative refers to a compound with hydrogen atoms (H) on anthracene substituted.

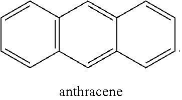 [SMILES: c1ccc2cc3ccccc3cc2c1]

For example, the first doping material and the second doping material are phosphorescent doping materials, and the third doping material is a fluorescent doping material or a phosphorescent doping material.

It will be noted that singlet excitons and triplet excitons generated after the phosphorescent doping material is excited may emit light when transitioning to the ground state, so that an internal quantum efficiency (IQE) of the light-emitting device 10 based on phosphorescent luminescence reaches 100%.

The fluorescent doping material may generate singlet excitons and triplet excitons in a ratio of 25:75 after being excited, where 25% of the singlet excitons emit fluorescence when transitioning to the ground state, and 75% of the triplet excitons do not emit light when transitioning to the ground state, but has a low cost and causes less pollution.

Setting the two first host materials each to be any of exciplexes, isomers and homologues, the two second host materials each to be any of exciplexes, isomers and homologues, and the two third host materials each to be any of exciplexes, isomers and homologues is beneficial to improving the utilization rate of excitons, thereby improving the efficiency of the light-emitting device 10.

Examples of common materials used for forming each film layer of the light-emitting device 10 are listed below.

For example, as shown in FIG. 1, the first electrode 19 is an anode, and is a single-layer transparent electrode made of a high work function material such as transparent oxide indium tin oxide (ITO) or indium zinc oxide (IZO), or may be a composite electrode formed by ITO/Ag/ITO, Ag/IZO, carbon nanotube/ITO (CNT/ITO), CNT/IZO, graphite oxide/ITO (GO/ITO), or GO/IZO.

For example, as shown in FIG. 1, the material of the hole injection layer 11 may be an inorganic oxide, such as molybdenum oxide, titanium oxide, vanadium oxide, rhenium oxide, ruthenium oxide, chromium oxide, zirconium oxide, hafnium oxide, tantalum oxide, silver oxide, tungsten oxide; or manganese oxide, or may be a dopant of strong electron absorption systems, such as F4TCNQ or HATCN. Alternatively, the hole transport material may be performed P-type doping, with a doping thickness of 5 nm to 20 nm, to form the hole injection layer 11 by co-evaporation. Alternatively, the material of the hole injection layer 11 may be PPDN. The structural formulas of F4TCNQ, HATCN and PPDN are as follows.

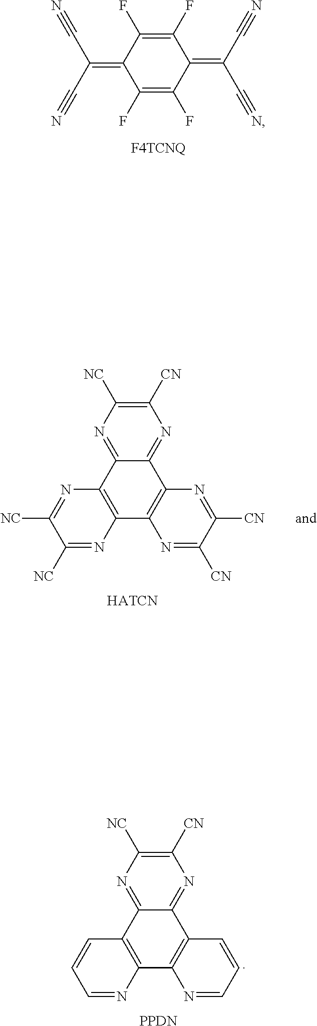

For example, as shown in FIG. 1, the material of the hole transport layer 12 has good hole transport properties and may be an arylamine or carbazole material, such as NPB, TPD, BAFLP or DFLDPBi. Alternatively, the material of the hole transport layer 12 may be TCTA or TAPC. The structural formulas of NPB, TCTA and TAPC are as follows.

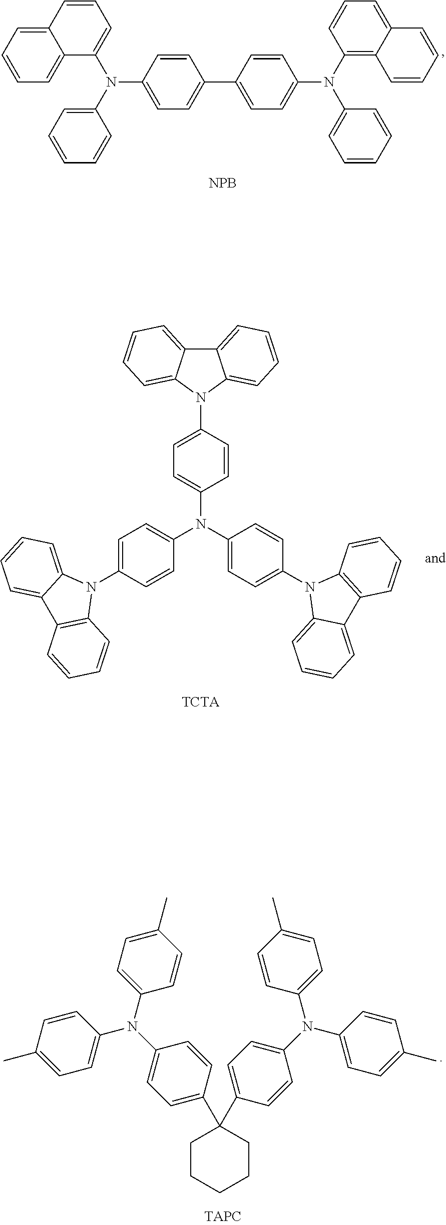

For example, as shown in FIG. 1, the material of the electron blocking layer 13 has good hole transport properties, and may use an arylamine or carbazole material, such as CBP or PCzPA.

For example, as shown in FIG. 1, the first sub-pixel film layer 14a is configured to emit red light. The first host material of the first sub-pixel film layer 14a may be selected from a DCM series of materials, such as DCM, DCJTB and DCJTI. The first doping material may be a metal complex, such as Ir(piq)2(acac), PtOEP or Ir(btp)2(acac). The structural formula of Ir(piq)2(acac) is as follows.

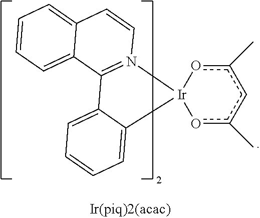

For example, as shown in FIG. 1, the second sub-pixel film layer 14b is configured to emit green light. The second host material of the second sub-pixel film layer 14b may be selected from coumarin dyes, quinacridone derivatives, polycyclic aromatic hydrocarbons, diamine anthracene derivatives and carbazole derivatives, such as DMQA, BA-NPB or Alq3. The second doping material may be a metal complex, such as Ir(ppy)3 or Ir(ppy)2(acac). The structural formula of Ir(ppy)3 is as follows.

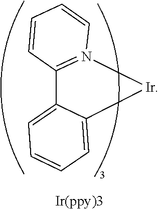

For example, as shown in FIG. 1, the third sub-pixel film layer 14c is configured to emit blue light. The third host material of the third sub-pixel film layer 14c may be selected from anthracene derivatives, ADN and MADN. The third doping material may be a pyrene derivative, a fluorene derivative, a perylene derivative, a styrylamine derivative, or a metal complexe, such as TBPe, BDAVBi, DPAVBi, or Flrpic. The structural formula of DPAVBi is as follows.

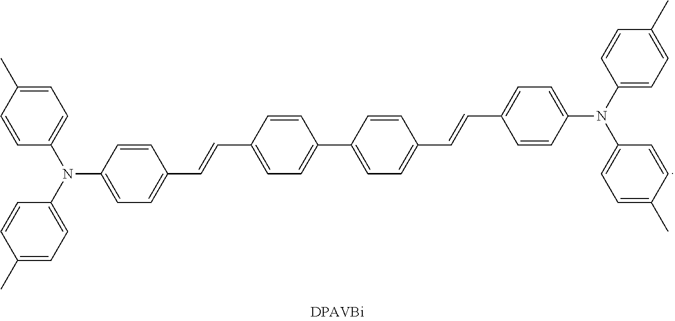 [SMILES: Cc1ccc(N(c2ccc(C)cc2)c2ccc(/C=C/c3ccc(-c4ccc(/C=C/c5ccc(N(c6ccc(C)cc6)c6ccc(C)cc6)cc5)cc4)cc3)cc2)cc1]

For example, as shown in FIG. 1, the hole generation layer 32 uses a hole-type material, such as NPB or TPD. The hole generation layer 32 may also be provided with a dopant. The dopant of the hole generation layer 32 may be HATCN, F4TCNQ, or the like. The fourth doping material of the electron generation layer 31 may be an alkali metal such as lithium (Li), sodium (Na), potassium (K) or cesium (Cs), or an alkaline-earth such as magnesium (Mg), strontium (Sr), barium (Ba) or radium (Ra) and oxides thereof.

For example, as shown in FIG. 1, the hole blocking layer 15 and the electron transport layer 16 are generally aromatic heterocyclic compounds, such as imidazole derivatives (e.g., benzimidazole derivatives, imidazopyridine derivatives and benzimidazophenanthridine derivatives), azine derivatives (e.g., pyrimidine derivatives and triazine derivatives), compounds containing a nitrogen six-membered ring structure (e.g., quinoline derivatives, isoquinoline derivatives and phenanthroline derivatives), BPhen or BCP; or include compounds having a phosphine oxide-based substituent on the heterocyclic ring, such as OXD-7, TAZ or p-EtTAZ; or include TPBi. The structural formulas of BPhen and TPBi are as follows.

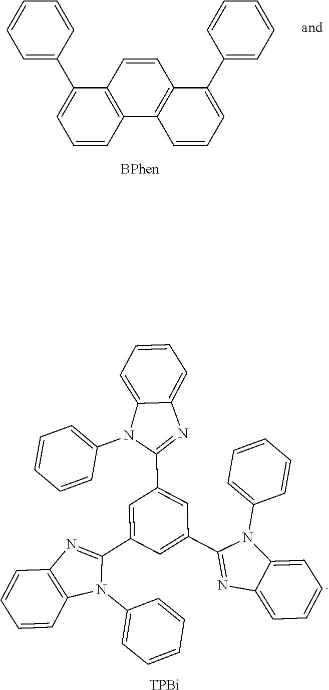

For example, as shown in FIG. 1, the material of the electron injection layer 17 is generally an alkali metal or a metal, or a compound thereof, such as LiF, Yb, Mg, Ca.

For example, as shown in FIG. 1, the light-emitting device 10 may be formed on a substrate 50, and the substrate 50 may select any transparent rigid or flexible substrate material, such as glass, or polyimide.

A method for manufacturing the light-emitting device 10 is described below.

For example, by considering an example of manufacturing the light-emitting device 10 shown in FIG. 3, the method for manufacturing the light-emitting device 10 includes steps S1 to S4. The manufacturing steps are shown in FIG. 4, and the structure refers to FIG. 3.

In S1, a first light-emitting unit 1 is formed on a substrate 50 with a first electrode 19.

For example, the first light-emitting unit 1 includes a hole injection layer 11, a hole transport layer 12, an electron blocking layer 13, a light-emitting layer 14 and a hole blocking layer 15. The electron blocking layer 13 includes a second electron blocking layer 13b and a first electron blocking layer 13a. The light-emitting layer 14 includes a first sub-pixel film layer 14a, a second sub-pixel film layer 14b and a third sub-pixel film layer 14c.

For example, before the first light-emitting unit 1 is formed on the substrate 50 with the first electrode 19, the method further includes a step S0. The step S0 include: cleaning the substrate 50 with the first electrode 19.

For example, the first electrode 19 is an anode, and the material of the first electrode 19 is indium tin oxide (ITO).

For example, the substrate 50 uses a glass substrate.

For example, the glass substrate with ITO is ultrasonically treated in a cleaning agent, rinsed in deionized water, ultrasonically degreased in an acetone-ethanol mixed solvent, and baked in a clean environment until the water is completely removed.

In S2, a charge generation unit 3 is formed on a side of the first light-emitting unit 1 away from the substrate 50.

For example, the charge generation unit 3 includes an electron generation layer 31 and a hole generation layer 32.

In S3, a second light-emitting unit 2 is formed on a side of the charge generation unit 3 away from the first light-emitting unit 1.

For example, the second light-emitting unit 2 includes a hole transport layer 12, an electron blocking layer 13, a light-emitting layer 14, a hole blocking layer 15, an electron transport layer 16 and an electron injection layer 17. The electron blocking layer 13 includes a second electron blocking layer 13b and a first electron blocking layer 13a. The light-emitting layer 14 includes a first sub-pixel film layer 14a, a second sub-pixel film layer 14b and a third sub-pixel film layer 14c.

In S4, a second electrode 20 is formed on a side of the second light-emitting unit 2 away from the charge generation unit 3.

For example, the material of the second electrode 20 uses a MgAg alloy, where a mass ratio of magnesium (Mg) to silver (Ag) is 1:9.

For example, the second electrode 20 is formed by evaporation.

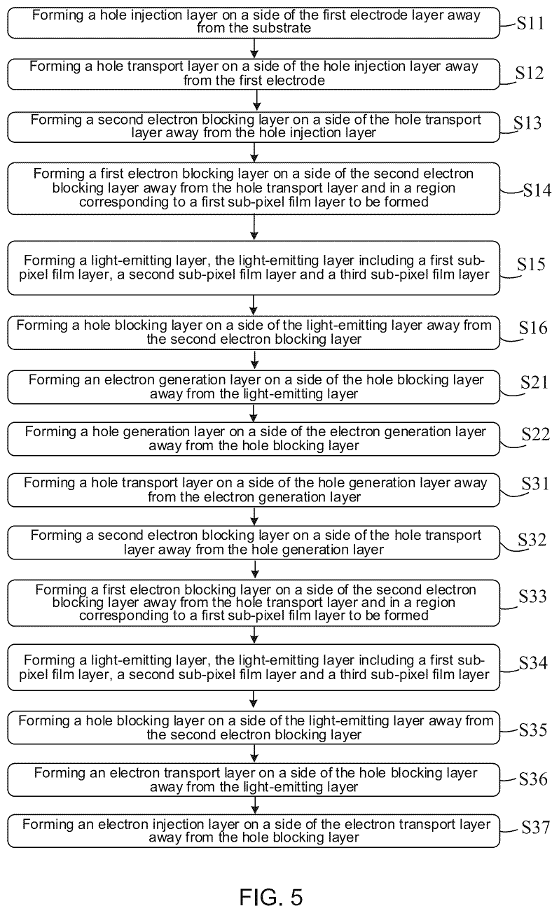

The steps for forming the first light-emitting unit 1 are described below. The specific steps of forming the first light-emitting unit 1 on the substrate 50 with the first electrode 19 in step S1 include S11 to S16, which is shown in FIG. 5, and the structure may refer to FIG. 3.

In S11, a hole injection layer 11 is formed on a side of the first electrode layer 19 away from the substrate 50.

For example, the material of the hole injection layer 11 includes HATCN and NPB, where a mass proportion of NPB in the hole injection layer 11 is 5%. For the structures of HATCN and NPB, reference may be made to the above contents, and details are not repeated here.

For example, the above substrate 50 with the first electrode 19 is placed in a vacuum chamber, evacuated to 1×10⁻⁵ Pa to 1×10⁻⁶ Pa, and HATCN and NPB are co-evaporated on a side of the first electrode 19 away from the substrate 50 in vacuum, so as to form the hole injection layer 11.

In S12, a hole transport layer 12 is formed on a side of the hole injection layer 11 away from the first electrode 19.

For example, the material of the hole transport layer 12 is N,N′-di(1-naphthyl)-N,N′-diphenyl-(1,1′-biphenyl)-4,4′-diamine (NPB).

For example, NPB is evaporated on a side of the hole injection layer 11 away from the first electrode 19, so as to form the hole transport layer 12.

In S13, a second electron blocking layer 13b is formed on a side of the hole transport layer 12 away from the hole injection layer 11.

For example, the structural formula of the material of the second electron blocking layer 13b is as shown in the following formula, represented by (13b-1).

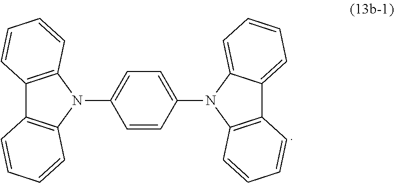 [SMILES: c1ccc2c(c1)c1ccccc1n2-c1ccc(-n2c3ccccc3c3ccccc32)cc1]

For example, a material as shown in the structural formula (13b-1) is evaporated on a side of the hole transport layer 12 away from the hole injection layer 11, so as to form the second electron blocking layer 13b.

In S14, a first electron blocking layer 13a is formed on a side of the second electron blocking layer 13b away from the hole transport layer 12 and in a region corresponding to a first sub-pixel film layer 14a to be formed.

For example, the material of the first electron blocking layer 13a uses TCTA. Regarding the structural formula of TCTA, reference may be made to the above contents, and details are not repeated here.

For example, TCTA is evaporated on a side of the second electron blocking layer 13b away from the hole transport layer 12 and in a region where the first sub-pixel film layer 14a to be formed is located, so as to form the first electron blocking layer 13a.

In S15, a light-emitting layer 14 is formed. The light-emitting layer 14 includes a first sub-pixel film layer 14a, a second sub-pixel film layer 14b and a third sub-pixel film layer 14c.

For example, the first sub-pixel film layer 14a is configured to emit red light. The material of the first sub-pixel film layer 14a includes two first host materials, and the two first host materials are materials as shown in the structural formulas (RH-1) and (RH-2). The two first host materials, as shown in the structural formulas (RH-1) and (RH-2), may form exciplexes. The material of the first sub-pixel film layer 14a further includes a first doping material. The first doping material uses Ir(piq)2(acac). As for the structural formula of Ir(piq)2(acac), reference may be made to the above contents, and details are not repeated here.

For example, a mixture material of the two first host materials as shown in the structural formulas (RH-1) and (RH-2) and Ir(piq)2(acac) is evaporated on a side of the first electron blocking layer 13a away from the second electron blocking layer 13b, so as to form the first sub-pixel film layer 14a.

For example, the second sub-pixel film layer 14b is configured to emit green light. The material of the second sub-pixel film layer 14b includes two second host materials, and the two second host materials are materials as shown in the structural formulas (GH-1) and (GH-2). The two second host materials, as shown in the structural formulas (GH-1) and (GH-2), may form exciplexes. The second sub-pixel film layer 14b further includes a second doping material. The second doping material uses Ir(ppy)3. As for the structural formula of the second doping material, reference may be made to the above contents, and details are not repeated here.

For example, a mixture material of the materials as shown in the structural formulas (GH-1) and (GH-2) and Ir(ppy)3 is evaporated on a side of the second electron blocking layer 13b away from the hole transport layer 12 and in a region where the second sub-pixel film layer 14b to be formed is located, so as to form the second sub-pixel film layer 14b.

For example, the third sub-pixel film layer 14c is configured to emit blue light. The third sub-pixel film layer 14c includes a third host material, and the third host material is a material as shown in the structural formula (BH-1). As for the structural formula shown in (BH-1), reference may be made to the above contents, and details are not repeated here. The third sub-pixel film layer 14c further includes a third doping material, and the structure of the third doping material is as shown in the following formula, represented by (BD-1).

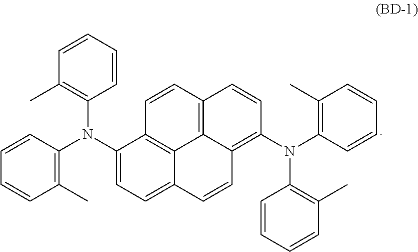 [SMILES: Cc1ccccc1N(c1ccccc1C)c1ccc2ccc3c(N(c4ccccc4C)c4ccccc4C)ccc4ccc1c2c43]

For example, a mixture material of the material as shown in the structural formula (BH-1) and the material as shown in the structural formula (BD-1) is evaporated on a side of the second electron blocking layer 13b away from the hole transport layer 12 and in a region where the third sub-pixel film layer 14c to be formed is located, so as to form the third sub-pixel film layer 14c.

The first sub-pixel film layer 14a, the second sub-pixel film layer 14b and the third sub-pixel film layer 14c form the light-emitting layer 14.

In S16, a hole blocking layer 15 is formed on a side of the light-emitting layer 14 away from the second electron blocking layer 13b.

For example, the material of the hole blocking layer 15 uses a material as shown in the following structural formula, represented by (HBL-1).

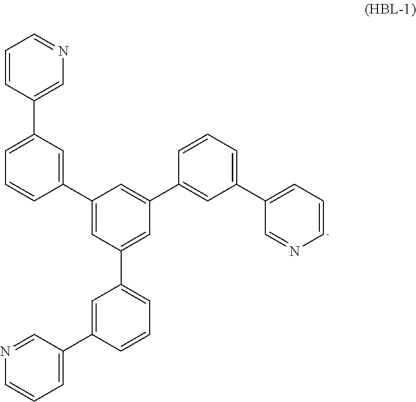 [SMILES: c1cncc(-c2cccc(-c3cc(-c4cccc(-c5cccnc5)c4)cc(-c4cccc(-c5cccnc5)c4)c3)c2)c1]

For example, the material as shown in the structural formula (HBL-1) is evaporated on a side of the light-emitting layer 14 away from the second electron blocking layer 13b, so as to form the hole blocking layer 15.

It can be understood that after the hole blocking layer 15 is formed, the first light-emitting unit 1 of the light-emitting device 10 is obtained.

The steps for forming the charge generation unit 3 are described below. The specific steps of forming the charge generation unit 3 on a side of the first light-emitting unit 1 away from the substrate 50 in step S2 include S21 to S22, which is shown in FIG. 5, and the structure may refer to FIG. 3.

In S21, an electron generation layer 31 is formed on a side of the hole blocking layer 15 away from the light-emitting layer 14.

For example, the material of the electron generation layer 31 uses a mixture material of a material as shown in the structural formula (3-6) and a metal ytterbium (Yb), and a mass proportion of the metal ytterbium (Yb) in the electron generation layer 31 is 1%. The structural formula shown in (3-6) may refer to the above contents, and details are not repeated here.

For example, a mixture material of the material as shown in the structural formula (3-6) and the metal ytterbium (Yb) is deposited on a side of the hole blocking layer 15 away from the light-emitting layer 14, so as to form the electron generation layer 31.

In S22, a hole generation layer 32 is formed on a side of the electron generation layer 31 away from the hole blocking layer 15.

For example, the hole generation layer 32 uses a mixture material of a material as shown in the structural formula (PCGL-1) and a triaxial alkali compound. The mass proportion of the triaxial alkali compound in the hole generation layer 32 is 5%. As for the structure of the triaxial alkali compound, reference may be made to the above contents, and details are not repeated here. The structural formula of (PCGL-1) is as

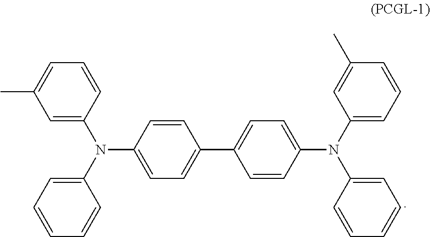 [SMILES: Cc1cccc(N(c2ccccc2)c2ccc(-c3ccc(N(c4ccccc4)c4cccc(C)c4)cc3)cc2)c1]

It can be understood that after the hole generation layer 32 is formed, the charge generation unit 3 of the light-emitting device 10 is obtained.

The steps for forming the second light-emitting unit 2 are described below. The specific steps of forming the second light-emitting unit 2 on a side of the charge generation unit 3 away from the first light-emitting unit 1 in step S3 include S31 to S37, which is shown in FIG. 5, and the structure may refer to FIG. 3.

In S31, a hole transport layer 12 is formed on a side of the hole generation layer 32 away from the electron generation layer 31.

The step of forming the hole transport layer 12 of the second light-emitting unit 2 may refer to the step of forming the hole transport layer 12 of the first light-emitting unit 1 in step S12, and details are not repeated here.

In S32, a second electron blocking layer 13b is formed on a side of the hole transport layer 12 away from the hole generation layer 32.

The step of forming the second electron blocking layer 13b of the second light-emitting unit 2 may refer to the step of forming the second electron blocking layer 13b of the first light-emitting unit 1 in step S13, and details are not repeated here.

In S33, a first electron blocking layer 13a is formed on a side of the second electron blocking layer 13b away from the hole transport layer 12 and in a region corresponding to a first sub-pixel film layer 14a to be formed.

The step of forming the first electron blocking layer 13a of the second light-emitting unit 2 may refer to the step of forming the first electron blocking layer 13a of the first light-emitting unit 1 in step S14, and details are not repeated here.

In S34, a light-emitting layer 14 is formed. The light-emitting layer 14 includes a first sub-pixel film layer 14a, a second sub-pixel film layer 14b and a third sub-pixel film layer 14c.

The step of forming the light-emitting layer 14 of the second light-emitting unit 2 may refer to the step of forming the light-emitting layer 14 of the first light-emitting unit 1 in step S15, and details are not repeated here.

In S35, a hole blocking layer 15 is formed on a side of the light-emitting layer 14 away from the second electron blocking layer 13b.

The step of forming the hole blocking layer 15 of the second light-emitting unit 2 may refer to the step of forming the hole blocking layer 15 of the first light-emitting unit 1 in step S16, and details are not repeated here.

In S36, an electron transport layer 16 is formed on a side of the hole blocking layer 15 away from the light-emitting layer 14.

For example, the material of the electron transport layer 16 is a mixture of TPBi and LiQ. The structural formulas of TPBi and LiQ refer to the above contents, and details are not repeated here.

For example, a mixture of TPBi and LiQ is evaporated on a side of the hole blocking layer 15 away from the light-emitting layer 14 in vacuum, so as to form the electron transport layer 16.

In S37, an electron injection layer 17 is formed on a side of the electron transport layer 16 away from the hole blocking layer 15.

For example, the material of the electron injection layer 17 uses the metal ytterbium (Yb).

For example, the metal ytterbium (Yb) is evaporated on a side of the electron transport layer 16 away from the hole blocking layer 15 in vacuum, so as to form the electron injection layer 17.

It can be understood that after the electron injection layer 17 is formed, the second light-emitting unit 2 of the light-emitting device 10 is obtained.

It will be noted that the film layers with the same functions of the first light-emitting unit 1 and the second light-emitting unit 2 of the light-emitting device 10 formed by the above manufacturing method have the same materials. For example, the hole transport layer 12 of the first light-emitting unit 1 and the hole transport layer 12 of the second light-emitting unit 2 have the same materials, the second electron blocking layer 13b of the first light-emitting unit 1 and the second electron blocking layer 13b of the second light-emitting unit 2 have the same materials, the first electron blocking layer 13a of the first light-emitting unit 1 and the first electron blocking layer 13a of the second light-emitting unit 2 have the same materials, and the hole blocking layer 15 of the first light-emitting unit 1 and the hole blocking layer 15 of the second light-emitting unit 2 have the same materials. The embodiments of the present disclosure are not limited thereto.

It can be understood that the film layers with the same functions of the first light-emitting unit 1 and the second light-emitting unit 2 of the light-emitting device 10 may have different materials. Moreover, the first type of functional layer 21 and the second type of functional layer 41 included in the first light-emitting unit 1 and the second light-emitting unit 2 may have the same or different structures and the same or different materials, which is not limited here.

Voltages, luminous efficiencies and device lives of the light-emitting devices 10 formed using different materials in different embodiments and comparative example are compared below.

The embodiments include Embodiment 4 to Embodiment 9. In the following comparative example and embodiments, the structures of the light-emitting devices 10 and the test conditions of the light-emitting devices 10 are the same.

The difference is that the hole blocking layers 15 (denoted as HBL), the first electron blocking layers 13a (denoted as REBL), the second electron blocking layers 13b (denoted as GEBL), the hole generation layers 32 (denoted PCGL), the electron transport layer 16 (denoted as ETL) and the hole transport layers 12 (denoted as HTL) used in the comparative example and embodiments have not exactly the same materials.

In Embodiment 4, the material of the hole blocking layer 15 (denoted as HBL) has a structural formula as shown in (2-1), the material of the first electron blocking layer 13a (denoted as REBL) has a structural formula as shown in (1-13), the material of the second electron blocking layer 13b (denoted as GEBL) has a structural formula as shown in (1-1), the material of the hole generation layer 32 (denoted as PCGL) has a structural formula as shown in (1-7), the material of the electron transport layer 16 (denoted as ETL) has a structural formula as shown in (2-8), and the material of the hole transport layer 12 (denoted as HTL) has a structural formula as shown in (1-5).

In Embodiment 5, the material of the hole blocking layer 15 (denoted as HBL) has a structural formula as shown in (2-1), the material of the first electron blocking layer 13a (denoted as REBL) has a structural formula as shown in (1-13), the material of the second electron blocking layer 13b (denoted as GEBL) uses comparative GEBL, the material of the hole generation layer 32 (denoted as PCGL) has a structural formula as shown in (1-7), the material of the electron transport layer 16 (denoted as ETL) has a structural formula as shown in (2-8), and the material of the hole transport layer 12 (denoted as HTL) has a structural formula as shown in (1-5).

For example, the comparative GEEL may use an arylamine or carbazole material, such as CBP or PCzPA. That is, the comparative GEBL uses a material not containing a compound centered on an sp3 hybridized carbon atom.

In Embodiment 6, the material of the hole blocking layer 15 (denoted as HBL) has a structural formula as shown in (2-1), the material of the first electron blocking layer 13a (denoted as REBL) has a structural formula as shown in (1-13), the material of the second electron blocking layer 13b (denoted as GEBL) uses comparative GEBL, the material of the hole generation layer 32 (denoted as PCGL) uses comparative PCGL, the material of the electron transport layer 16 (denoted as ETL) has a structural formula as shown in (2-8), and the material of the hole transport layer 12 (denoted as HTL) has a structural formula as shown in (1-5).

For example, the comparative PCGL may use N,N′-di(1-naphthyl)-N,N′-diphenyl-(1,1′-biphenyl)-4,4′-diamine (NPB). That is, the comparative PCGL uses a material not containing a compound centered on an sp3 hybridized carbon atom.

In Embodiment 7, the material of the hole blocking layer 15 (denoted as HBL) has a structural formula as shown in (2-1), the material of the first electron blocking layer 13a (denoted as REBL) uses comparative REBL, the material of the second electron blocking layer 13b (denoted as GEBL) uses comparative GEBL, the material of the hole generation layer 32 (denoted as PCGL) uses comparative PCGL, the material of the electron transport layer 16 (denoted as ETL) has a structural formula as shown in (2-8), and the material of the hole transport layer 12 (denoted as HTL) has a structural formula as shown in (1-5).

For example, the comparative REBL uses an arylamine or carbazole material, such as CBP or PCzPA. That is, the comparative REBL uses a material not containing a compound centered on an sp3 hybridized carbon atom.

In Embodiment 8, the material of the hole blocking layer 15 (denoted as HBL) uses comparative HBL, the material of the first electron blocking layer 13a (denoted as REBL) has a structural formula as shown in (1-13), the material of the second electron blocking layer 13b (denoted as GEBL) has a structural formula as shown in (1-1), the material of the hole generation layer 32 (denoted as PCGL) has a structural formula as shown in (1-7), the material of the electron transport layer 16 (denoted as ETL) uses comparative ETL, and the material of the hole transport layer 12 (denoted as HTL) has a structural formula as shown in (1-5).

For example, the comparative HBL uses BPhen, the structural formula of BPhen may refer to the above contents, and details are not repeated here. The comparative ETL uses a material not containing a compound centered on an sp3 hybridized carbon atom. The comparative ETL uses TPBi, the structural formula of TPBi may refer to the above contents, and details are not repeated here. The comparative ETL uses a material not containing a compound centered on an sp3 hybridized carbon atom.

In Embodiment 9, the material of the hole blocking layer 15 (denoted as HBL) has a structural formula as shown in (2-1), the material of the first electron blocking layer 13a (denoted as REBL) uses comparative REBL, the material of the second electron blocking layer 13b (denoted as GEBL) uses comparative GEBL, the material of the hole generation layer 32 (denoted as PCGL) uses comparative PCGL, the material of the electron transport layer 16 (denoted as ETL) uses comparative ETL, and the material of the hole transport layer 12 (denoted as HTL) uses comparative HTL.

For example, the comparative HTL uses NPB, the structural formula of NPB may refer to the above contents, and details are not repeated here. The comparative HTL uses a material not containing a compound centered on an sp3 hybridized carbon atom.

In Comparative Example 1, the material of the hole blocking layer 15 (denoted as HBL) uses comparative HBL, the material of the first electron blocking layer 13a (denoted as REBL) uses comparative REBL, the material of the second electron blocking layer 13b (denoted as GEBL) uses comparative GEBL, the material of the hole generation layer 32 (denoted as PCGL) uses comparative PCGL, the material of the electron transport layer 16 (denoted as ETL) uses comparative ETL, and the material of the hole transport layer 12 (denoted as HTL) uses comparative HTL.

In order to more clearly describe the difference between structural formulas of materials of the hole blocking layers 15 (denoted as HBL), the first electron blocking layers 13a (denoted as REBL), the second electron blocking layers 13b (denoted as EBL), the hole generation layers 32 (denoted PCGL), the electron transport layer 16 (denoted as ETL) and the hole transport layers 12 (denoted as HTL) used in the embodiments and comparative example, Table 2 is used to more clearly show the structural formulas of the materials of the hole blocking layers 15 (denoted as HBL), the first electron blocking layers 13a (denoted as REBL), the second electron blocking layers 13b (denoted as GEBL), the hole generation layers 32 (denoted PCGL), the electron transport layer 16 (denoted as ETL) and the hole transport layers 12 (denoted as HTL) used in the embodiments and comparative example.

TABLE 2

| | HBL | REBL | GEBL | PCGL | ETL | HTL |
|---|---|---|---|---|---|---|
| Embodiment | (2-1) | (1-13) | (1-1) | (1-7) | (2-8) | (1-5) |
| 4 | | | | | | |
| Embodiment | (2-1) | (1-13) | Comparative | (1-7) | (2-8) | (1-5) |
| 5 | | | GEBL | | | |
| Embodiment | (2-1) | (1-13) | Comparative | Comparative | (2-8) | (1-5) |
| 6 | | | GEBL | PCGL | | |
| Embodiment | (2-1) | Comparative | Comparative | Comparative | (2-8) | (1-5) |
| 7 | | REBL | GEBL | PCGL | | |
| Embodiment | Comparative | (1-13) | (1-1) | (1-7) | Comparative | (1-5) |
| 8 | HBL | | | | ETL | |
| Embodiment | (2-1) | Comparative | Comparative | Comparative | Comparative | Comparative |
| 9 | | REBL | GEBL | PCGL | ETL | HTL |
| Comparative | Comparative | Comparative | Comparative | Comparative | Comparative | Comparative |
| Example 1 | HBL | REBL | GEBL | PCGL | ETL | HTL |

The structural formulas of the materials as shown in (2-1), (1-13), (1-1), (1-7), (2-8) and (1-5) refer to the above contents, and details are not repeated here.

Based on the above materials, the materials used in Embodiment 4 to Embodiment 9 and Comparative Example 1 are made into corresponding film layers, and performance of the voltages (V), luminous efficiencies (cd/A) and device lives (h) of the light-emitting devices 10 in Embodiment 4 to Embodiment 9 and Comparative Example 1 are tested. The data structure is provided based on Comparative Example 1 as a reference, and the test results are shown in Table 3 below.

TABLE 3

| | | Luminous | Device |
|---|---|---|---|
| | Voltage | efficiency | life |
| Embodiment | 92% | 119% | 152% |
| 4 | | | |
| Embodiment | 94% | 117% | 133% |
| 5 | | | |
| Embodiment | 97% | 108% | 128% |
| 6 | | | |
| Embodiment | 96% | 106% | 111% |
| 7 | | | |
| Embodiment | 96% | 105% | 109% |
| 8 | | | |
| Embodiment | 97% | 102% | 103% |
| 9 | | | |
| Comparative | 100% | 100% | 100% |
| Example 1 | | | |

It can be seen from Table 3 that taking the test data in Comparative Example 1 as a reference, the data of voltage, efficiency and life in Comparative Example 1 are set to 100%. The efficiencies and lives of the light-emitting devices 10 in Embodiment 4 to Embodiment 9 are significantly improved compared with Comparative Example 1. Therefore, the functional layer(s) of the light-emitting device 10 are formed using the material containing the compound centered on the sp3 hybridized carbon atom, so that the photoelectric performance of the light-emitting device 10 is improved.

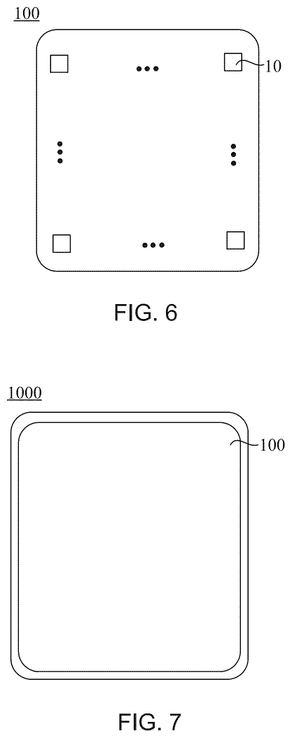

Some embodiments of the present disclosure further provide a light-emitting substrate 100. As shown in FIG. 6, the light-emitting substrate 100 includes the light-emitting devices 10 as described in any of the above embodiments.

Beneficial effects of the above light-emitting substrate 100 are the same as the beneficial effects of the light-emitting device 10 provided in the above embodiments of the present disclosure, and details are not described here again.

Some embodiments of the present disclosure provide a light-emitting apparatus 1000. As shown in FIG. 7, the light-emitting apparatus 1000 includes the light-emitting substrate 100 as described above. Of course, the light-emitting apparatus 1000 may further include other components. For example, the light-emitting apparatus 1000 may include a circuit for providing the light-emitting substrate 100 with electrical signals to drive the light-emitting substrate 100 to emit light, the circuit may be called a control circuit and may include a circuit board electrically connected to the light-emitting substrate 100 and/or an integrated circuit (IC) electrically connected to the light-emitting substrate 100.

In some embodiments, the light-emitting apparatus 1000 may be a lighting apparatus. In this case, the light-emitting apparatus 1000 is used as a light source to achieve a lighting function. For example, the light-emitting apparatus 1000 may be a backlight module in a liquid crystal display apparatus, a lamp for internal or external lighting, or a variety of signal lamps.

In some other embodiments, the light-emitting apparatus 1000 may be a display apparatus. In this case, the light-emitting substrate 100 is a display substrate for achieving a function of displaying an image (i e., a picture). The light-emitting apparatus 1000 may include a display or a product including the display. The display may be a flat panel display (FPD), a micro display, or the like. If classified according to whether a user may see a scene behind the display, the display may be a transparent display or an opaque display. If classified according to whether the display can be bent or curled, the display may be a flexible display or a common display (which may be referred to as a rigid display). For example, the product including the display may include a computer display, a television, a billboard, a laser printer with a display function, a telephone, a mobile phone, a personal digital assistant (PDA), a laptop computer, a digital camera, a portable camcorder, a viewfinder, a vehicle, a large-area wall, a screen in a theater or a sign in a stadium.

Beneficial effects of the above light-emitting apparatus 1000 are the same as the beneficial effects of the light-emitting device 10 provided in the above embodiments of the present disclosure, and details are not described here again.

The foregoing descriptions are merely specific implementations of the present disclosure, but the protection scope of the present disclosure is not limited thereto. Changes or replacements that any person skilled in the art could conceive of within the technical scope of the present disclosure shall be included in the protection scope of the present disclosure. Therefore, the protection scope of the present disclosure shall be subject to the protection scope of the claims.

1. A functional layer material, comprising a compound centered on an sp3 hybridized carbon atom, wherein
the compound centered on the sp3 hybridized carbon atom includes a first type of compound, and the first type of compound is selected from any one of structures shown in a following general formula (I): 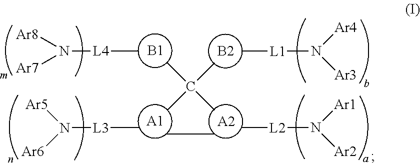
wherein values of a, b, m and n are each independently selected from any one of 0, 1, 2, 3 and 4, and at least one of a, b, m and n is not 0;
A1 and A2 are same or different, and are each independently selected from any one of a substituted or unsubstituted trivalent aryl group, a substituted or unsubstituted fused ring trivalent aryl group, and a substituted or unsubstituted fused ring trivalent heteroaryl group;
B1 and B2 are same or different, and are each independently selected from any one of a substituted or unsubstituted alkylene group, a substituted or unsubstituted arylene group, and a substituted or unsubstituted heteroarylene group;
L1, L2, L3 and L4 are same or different, and are each independently selected from any one of a single bond, a substituted or unsubstituted phenylene group, a substituted or unsubstituted biphenylene group, a substituted or unsubstituted terphenylene group, a substituted or unsubstituted fluorenyl group, a substituted or unsubstituted adamantyl group, and a substituted or unsubstituted heteroarylene group; and
Ar1, Ar2, Ar3, Ar4, Ar5, Ar6, Ar7 and Ar8 are same or different, and are each independently selected from any one of a substituted or unsubstituted alkyl group, a substituted or unsubstituted aryl group, a substituted or unsubstituted heteroaryl group, a substituted of unsubstituted fused ring aryl group and a substituted or unsubstituted fused ring heteroaryl group.

2. (canceled)

3. The functional layer material according to claim 1, wherein
A1 and A2 are the same or different, and are each independently selected from any one of a substituted or unsubstituted phenyl group, a substituted or unsubstituted naphthyl group, a substituted or unsubstituted phenanthrenyl group, a substituted or unsubstituted benzofuranyl group, a substituted or unsubstituted benzothienyl group, a substituted or unsubstituted dibenzofuranyl group dibenzofuran and a substituted or unsubstituted dibenzothienyl group; and
B1 and B2 are the same or different, and are each independently selected from any one of a substituted or unsubstituted methylene group, a substituted or unsubstituted adamantyl group, and a substituted or unsubstituted cyclohexyl group; and/or
B1 and B2 are capable of bonding to be a ring.

4-5. (canceled)

6. A light-emitting device, comprising at least two light-emitting units; each light-emitting unit in the at least two light-emitting units including a light-emitting layer, a first type of functional layer disposed on a side of the light-emitting layer, and a second type of functional layer disposed on another side of the light-emitting layer, wherein
the first type of functional layer includes a plurality of hole transport functional layers, and at least two hole transport functional layers in the plurality of hole transport functional layers each include the first type of compound according to claim 1; and
the second type of functional layer includes an electron transport functional layer, and the electron transport functional layer includes a second type of compound; or the second type of functional layer includes a plurality of electron transport functional layers, and at least two electron transport functional layers in the plurality of electron transport functional layers each include the second type of compound; wherein
the second type of compound is selected from any one of structures shown in a following general formula (II): 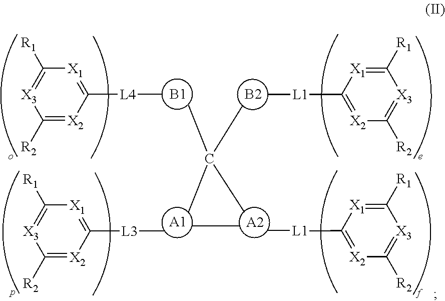
wherein values of e, f, o and p are each independently selected from any one of 0, 1, 2, 3 and 4, and at least one of e, f, o and p is not 0;
X₁, X₂ and X₃ are same or different, and are each independently selected from any one of —CR₃ and N, and at least one of X₁, X₂ and X₃ is N; and
R₁, R₂ and R₃ are same or different, and are each independently selected from any one of a substituted or unsubstituted aryl group and a substituted or unsubstituted heteroaryl group.

7. The light-emitting device according to claim 6, further comprising a charge generation unit disposed between two adjacent light-emitting units in the at least two light-emitting units, wherein the charge generation unit includes a hole generation layer and an electron generation layer; and
the hole generation layer includes two materials, and at least one of the two materials is the first type of compound.

8. The light-emitting device according to claim 7, wherein the electron generation layer includes a fourth host material, and the fourth host material is selected from any one of structures shown in following general formula (III); 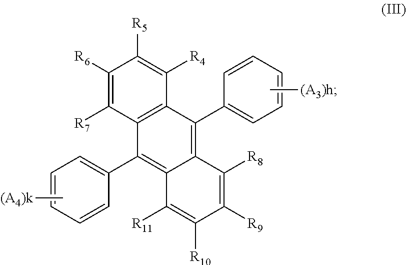
wherein R₄, R₅, R₆, R₇, R₈, R₉, R₁₀, R₁₁, A₃ and A₄ are same or different, and are each independently selected from a phosphonoxy group, H, D, F, a substituted or unsubstituted C1 to C18 alkyl group, a substituted or unsubstituted C6 to C60 aryl group, or a substituted or unsubstituted C3 to C60 heteroaryl group; at least one of R₄, R₅, R₆, R₇, R₈, R₉, R₁₀, R₁₁, A₃ and A₄ is the phosphonoxy group; and
values of k and h are each independently selected from any one of 0, 1, 2, 3, 4 and 5.

9. The light-emitting device according to claim 8, wherein two adjacent ones of R₄, R₅, R₆, R₇, R₈, R₉, R₁₀ and R₁₁ are capable of bonding to be a ring;
h is greater than or equal to 2 (h≥2), adjacent A₃ are capable of bonding to be a ring; and
k is greater than or equal to 2 (k≥2), adjacent A₄ are capable of bonding to be a ring.

10. The light-emitting device according to claim 6, wherein the light-emitting layer includes a first sub-pixel film layer, a second sub-pixel film layer and a third sub-pixel film layer; the first sub-pixel film layer, the second sub-pixel film layer and the third sub-pixel film layer are arranged in a first direction; and
the first sub-pixel film layer is configured to emit one of red light, blue light and green light, the second sub-pixel film layer is configured to emit another of red light, blue light and green light, and the third sub-pixel film layer is configured to emit a last one of red light, blue light and green light.

11. The light-emitting device according to claim 10, wherein the first type of functional layer includes a hole transport layer and a plurality of electron blocking layers disposed between the hole transport layer and the light-emitting layer;
at least two of the hole transport layer and the plurality of electron blocking layers each contain the first type of compound; and
the second type of functional layer includes the electron transport functional layer, the electron transport functional layer is a hole blocking layer, and the hole blocking layer contains the second type of compound; or the second type of functional layer includes the plurality of electron transport functional layers, the plurality of electron transport functional layers include a hole blocking layer and an electron transport layer, and the hole blocking layer and the electron transport layer each contain the second type of compound.

12. The light-emitting device according to claim 11, wherein the plurality of electron blocking layers include a first electron blocking layer, a second electron blocking layer and a third electron blocking layer; the first electron blocking layer, the second electron blocking layer and the third electron blocking layer are arranged in the first direction; or
the plurality of electron blocking layers include a first electron blocking layer and a second electron blocking layer; the second electron blocking layer is disposed between the light-emitting layer and the hole transport layer; and the first electron blocking layer is disposed between the second electron blocking layer and the first sub-pixel film layer.

13. The light-emitting device according to claim 12, wherein the first sub-pixel film layer is configured to emit red light, and the first electron blocking layer is disposed between the first sub-pixel film layer and the hole transport layer; and
the first electron blocking layer and the hole transport layer each contain the first type of compound; or
the first sub-pixel film layer is configured to emit red light, and the first electron blocking layer is disposed between the first sub-pixel film layer and the hole transport layer; the second sub-pixel film layer is configured to emit green light, and the second electron blocking layer is disposed between the second sub-pixel film layer and the hole transport layer; and the first electron blocking layer and the second electron blocking layer each contain the first type of compound.

14. (canceled)

15. The light-emitting device according to claim 11, wherein the plurality of electron blocking layers include a first electron blocking layer, a second electron blocking layer and a third electron blocking layer;
the first sub-pixel film layer is configured to emit red light, and the first electron blocking layer is disposed between the first sub-pixel film layer and the hole transport layer;
the second sub-pixel film layer is configured to emit green light, and the second electron blocking layer is disposed between the second sub-pixel film layer and the hole transport layer; and
the third sub-pixel film layer is configured to emit blue light, and the third electron blocking layer is disposed between the third sub-pixel film layer and the hole transport layer; wherein
a specific surface area of the first electron blocking layer is smaller than a specific surface area of the second electron blocking layer, and the specific surface area of the first electron blocking layer is smaller than a specific surface area of the third electron blocking layer.

16. (canceled)

17. The light-emitting device according to claim 11, wherein the at least two light-emitting units include a first light-emitting unit and a second light-emitting unit;
the first light-emitting unit, the charge generation unit and the second light-emitting unit are stacked in sequence in a second direction; the second direction is perpendicular to the first direction;
the first type of functional layer, the light-emitting layer and the second type of functional layer of the first light-emitting unit are stacked in the second direction; the first type of functional layer of the first light-emitting unit further includes a hole injection layer, and the hole injection layer is disposed on a side of the hole transport layer away from the light-emitting layer; and
the first type of functional layer, the light-emitting layer and the second type of functional layer of the second light-emitting unit are stacked in the second direction; the second type of functional layer of the second light-emitting unit includes the hole blocking layer and the electron transport layer, the second type of functional layer of the second light-emitting unit further includes an electron injection layer, and the electron injection layer is disposed on a side of the electron transport layer away from the light-emitting layer.

18. The light-emitting device according to claim 17, wherein a sub-pixel film layer of the first light-emitting unit and a sub-pixel film layer of the second light-emitting unit emit light of same color; a difference between a wavelength of light emitted by the sub-pixel film layer of the first light-emitting unit and a wavelength of light emitted by the sub-pixel film layer of the second light-emitting unit is less than or equal to 20 nm; wherein
the sub-pixel film layer of the first light-emitting unit and the sub-pixel film layer of the second light-emitting unit each include any one of the first sub-pixel film layer, the second sub-pixel film layer and the third sub-pixel film layer.

19. The light-emitting device according to claim 17, wherein a ratio of mobility of the hole blocking layer of the first light-emitting unit to mobility of the hole blocking layer of the second light-emitting unit is less than or equal to 10 and greater than or equal to 0.1; and
a ratio of mobility of the hole transport layer of the first light-emitting unit to mobility of the hole transport layer of the second light-emitting unit is less than or equal to 10 and greater than or equal to 0.1.

20. The light-emitting device according to claim 17, further comprising a charge generation unit disposed between the first light-emitting unit and the second light-emitting unit, wherein the charge generation unit includes a hole generation layer and an electron generation layer;
a difference between a HOMO energy level of the hole generation layer and a HOMO energy level of the hole transport layer of the second light-emitting unit is less than or equal to 0.3 eV; and
a difference between a LUMO energy level of the electron generation layer and a LUMO energy level of the hole blocking layer of the first light-emitting unit is less than or equal to 0.5 eV.

21. The light-emitting device according to claim 7, wherein a dipole moment of the electron generation layer is greater than 4 D; and/or:
the electron generation layer further includes a fourth doping material, and the fourth doping material includes any one of alkali metals and oxides thereof, alkaline-earth metals and oxides thereof, and transition metals and oxides thereof.

22. (canceled)

23. The light-emitting device according to claim 10, wherein the first sub-pixel film layer includes at least one first host material and a first doping material, the at least one first host material includes two first host materials, and each of the two first host materials is any one of exciplexes, isomers and homologues;
the second sub-pixel film layer includes at least two second host materials and a second doping material, the at least two second host materials include two second host materials, and each of the two second host materials is any one of exciplexes, isomers and homologues; and
the third sub-pixel film layer includes at least one third host material and a third doping material, the at least one third host material includes two third host materials, and each of the two third host materials is any one of exciplexes, isomers and homologues; and a third host material in the at least one third host material contains an anthracene derivative.

24. The light-emitting device according to claim 7, further comprising a first electrode and a second electrode, wherein the at least two light-emitting units and the charge generation unit are disposed between the first electrode and the second electrode.

25. A light-emitting substrate, comprising the light-emitting device according to claim 6.

26. A light-emitting apparatus, comprising the light-emitting substrate according to claim 25.
